SEQUENCE LISTING (60 sequences)

SEQ ID NO: 1 - DNA, 21 bp, Artificial Sequence
  Description of Artificial Sequence Synthetic oligonucleotide
aacctttggagaagatactgt

SEQ ID NO: 2 - DNA, 21 bp, Artificial Sequence
  Description of Artificial Sequence Synthetic oligonucleotide
aatcacatttgagcttgatga

SEQ ID NO: 3 - DNA, 21 bp, Artificial Sequence
  Description of Artificial Sequence Synthetic oligonucleotide
aagttgctggttttgcaaagt

SEQ ID NO: 4 - DNA, 21 bp, Artificial Sequence
  Description of Artificial Sequence Synthetic oligonucleotide
aaggatgaggaaggcaattta

SEQ ID NO: 5 - DNA, 21 bp, Artificial Sequence
  Description of Artificial Sequence Synthetic oligonucleotide
aagctcctaattacactcaac

SEQ ID NO: 6 - DNA, 21 bp, Artificial Sequence
  Description of Artificial Sequence Synthetic oligonucleotide
aatgttacagggtttcatact

SEQ ID NO: 7 - DNA, 47 bp, Artificial Sequence
  Description of Artificial Sequence Synthetic primer
gaacatcgatgacaagcttaggtatcgatagacaacctgctcataaa

SEQ ID NO: 8 - DNA, 47 bp, Artificial Sequence
  Description of Artificial Sequence Synthetic primer
gaacatcgatgacaagcttaggtatcgatagaggatgggcatcagca

SEQ ID NO: 9 - DNA, 47 bp, Artificial Sequence
  Description of Artificial Sequence Synthetic primer
gaacatcgatgacaagcttaggtatcgatagtgttaaaaccagaagg

SEQ ID NO: 10 - DNA, 30 bp, Artificial Sequence
  Description of Artificial Sequence Synthetic primer
gaacatcgatgacaagcttaggtatcgata

SEQ ID NO: 11 - DNA, 30 bp, Artificial Sequence
  Description of Artificial Sequence Synthetic primer
gggaagttcaaggttacaagaatgtgagaa

SEQ ID NO: 12 - DNA, 30 bp, Artificial Sequence
  Description of Artificial Sequence Synthetic primer
cggtgtaagtgcagcccgtcttacaccgtg

SEQ ID NO: 13 - DNA, 30 bp, Artificial Sequence
  Description of Artificial Sequence Synthetic primer
ccttgaccggtgcaccacttttgatgatgt

SEQ ID NO: 14 - DNA, 21 bp, Artificial Sequence
  Description of Artificial Sequence Synthetic primer
gcatgaaattgcctggttcac

SEQ ID NO: 15 - DNA, 21 bp, Artificial Sequence
  Description of Artificial Sequence Synthetic primer
gcattcccctttgaaagtgtc

SEQ ID NO: 16 - DNA, 28 bp, Artificial Sequence
  Description of Artificial Sequence Synthetic primer
agctacgagcaccagacacccttcgaaa

SEQ ID NO: 17 - DNA, 29 bp, Artificial Sequence
  Description of Artificial Sequence Synthetic primer
aacatcgatgacaagcttaggtatcgata

SEQ ID NO: 18 - DNA, 21 bp, Artificial Sequence
  Description of Artificial Sequence Synthetic oligonucleotide
aagagactatttataacttgg

SEQ ID NO: 19 - DNA, 21 bp, Artificial Sequence
  Description of Artificial Sequence Synthetic oligonucleotide
aacgagtaactcgtccctctt

SEQ ID NO: 20 - DNA, 21 bp, Artificial Sequence
  Description of Artificial Sequence Synthetic oligonucleotide
aattgcataccgcaatgttct

SEQ ID NO: 21 - DNA, 21 bp, Artificial Sequence
  Description of Artificial Sequence Synthetic oligonucleotide
aacctacacctgaagaaccag

SEQ ID NO: 22 - DNA, 21 bp, Artificial Sequence
  Description of Artificial Sequence Synthetic oligonucleotide
aaggatgtgctggttatacac

SEQ ID NO: 23 - DNA, 21 bp, Artificial Sequence
  Description of Artificial Sequence Synthetic oligonucleotide
aaaggaccagtgactgatgtt

SEQ ID NO: 24 - DNA, 21 bp, Artificial Sequence
  Description of Artificial Sequence Synthetic oligonucleotide
aaggtgttgttgataccgatg

SEQ ID NO: 25 - DNA, 21 bp, Artificial Sequence
  Description of Artificial Sequence Synthetic oligonucleotide
aagcacgcattcttgtgcttg

SEQ ID NO: 26 - DNA, 21 bp, Artificial Sequence
  Description of Artificial Sequence Synthetic oligonucleotide
aaggataagtcagctcaatgc

SEQ ID NO: 27 - DNA, 21 bp, Artificial Sequence
  Description of Artificial Sequence Synthetic oligonucleotide
aactggcacactacttgtcga

SEQ ID NO: 28 - DNA, 21 bp, Artificial Sequence
  Description of Artificial Sequence Synthetic oligonucleotide
aactcctattcgtagttgaag

SEQ ID NO: 29 - DNA, 21 bp, Artificial Sequence
  Description of Artificial Sequence Synthetic oligonucleotide
aaggtgactatggtgatgctg

SEQ ID NO: 30 - DNA, 21 bp, Artificial Sequence
  Description of Artificial Sequence Synthetic oligonucleotide
aacctacctctccagctagga

SEQ ID NO: 31 - DNA, 21 bp, Artificial Sequence
  Description of Artificial Sequence Synthetic oligonucleotide
aagggctatcaacctatagat

SEQ ID NO: 32 - DNA, 21 bp, Artificial Sequence
  Description of Artificial Sequence Synthetic oligonucleotide
aatcacagatgctgttgattg

SEQ ID NO: 33 - DNA, 21 bp, Artificial Sequence
  Description of Artificial Sequence Synthetic oligonucleotide
aaccttacagagttgtagtac

SEQ ID NO: 34 - DNA, 21 bp, Artificial Sequence
  Description of Artificial Sequence Synthetic oligonucleotide
aagatgttaactgcactgatg

SEQ ID NO: 35 - DNA, 21 bp, Artificial Sequence
  Description of Artificial Sequence Synthetic oligonucleotide
aagagctggacaagtacttca

SEQ ID NO: 36 - DNA, 21 bp, Artificial Sequence
  Description of Artificial Sequence Synthetic oligonucleotide
aagaaacaggtacgttaatag

SEQ ID NO: 37 - DNA, 21 bp, Artificial Sequence
  Description of Artificial Sequence Synthetic oligonucleotide
aatagttaatagcgtacttct

SEQ ID NO: 38 - DNA, 21 bp, Artificial Sequence
  Description of Artificial Sequence Synthetic oligonucleotide
tgtgcgtactgctgcaatatt

SEQ ID NO: 39 - DNA, 21 bp, Artificial Sequence
  Description of Artificial Sequence Synthetic oligonucleotide
aaggagttcctgatcttctgg

SEQ ID NO: 40 - DNA, 21 bp, Artificial Sequence
  Description of Artificial Sequence Synthetic oligonucleotide
aacctagtaataggtttccta

SEQ ID NO: 41 - DNA, 21 bp, Artificial Sequence
  Description of Artificial Sequence Synthetic oligonucleotide
aatggcttgtattgtaggctt

SEQ ID NO: 42 - DNA, 21 bp, Artificial Sequence
  Description of Artificial Sequence Synthetic oligonucleotide
aattgtgaccagaccgctcat

SEQ ID NO: 43 - DNA, 21 bp, Artificial Sequence
  Description of Artificial Sequence Synthetic oligonucleotide
aagagatcactgtggctacat

SEQ ID NO: 44 - DNA, 21 bp, Artificial Sequence
  Description of Artificial Sequence Synthetic oligonucleotide
aaccgctaccgtattggaaac

SEQ ID NO: 45 - DNA, 21 bp, Artificial Sequence
  Description of Artificial Sequence Synthetic oligonucleotide
aaccagcttgagagcaaagtt

SEQ ID NO: 46 - DNA, 21 bp, Artificial Sequence
  Description of Artificial Sequence Synthetic oligonucleotide
aagcacattgacgcatacaaa

SEQ ID NO: 47 - DNA, 21 bp, Artificial Sequence
  Description of Artificial Sequence Synthetic oligonucleotide
aacagtacaacgtcactcaag

SEQ ID NO: 48 - DNA, 21 bp, Artificial Sequence
  Description of Artificial Sequence Synthetic oligonucleotide
aagactgatgaagctcagcct

SEQ ID NO: 49 - DNA, 21 bp, Artificial Sequence
  Description of Artificial Sequence Synthetic oligonucleotide
aagtactgttcatgctacagc

SEQ ID NO: 50 - DNA, 21 bp, Artificial Sequence
  Description of Artificial Sequence Synthetic oligonucleotide
aatgcatcaacgcatgtagaa

SEQ ID NO: 51 - DNA, 21 bp, Artificial Sequence
  Description of Artificial Sequence Synthetic oligonucleotide
aattattatgagatgttggct

SEQ ID NO: 52 - DNA, 21 bp, Artificial Sequence
  Description of Artificial Sequence Synthetic oligonucleotide
aaggtgacggcatttcaacac

SEQ ID NO: 53 - DNA, 21 bp, Artificial Sequence
  Description of Artificial Sequence Synthetic oligonucleotide
aaattactacagacactggta

SEQ ID NO: 54 - DNA, 21 bp, Artificial Sequence
  Description of Artificial Sequence Synthetic oligonucleotide
aaaatgctacattcttcatct

SEQ ID NO: 55 - DNA, 21 bp, Artificial Sequence
  Description of Artificial Sequence Synthetic oligonucleotide
aatacacacaatcgacggctc

SEQ ID NO: 56 - DNA, 21 bp, Artificial Sequence
  Description of Artificial Sequence Synthetic oligonucleotide
aaccttgcccatcaggaacat

SEQ ID NO: 57 - DNA, 21 bp, Artificial Sequence
  Description of Artificial Sequence Synthetic oligonucleotide
aacttgcactagcacacactt

SEQ ID NO: 58 - DNA, 21 bp, Artificial Sequence
  Description of Artificial Sequence Synthetic oligonucleotide
aagagctctactcgccacttt

SEQ ID NO: 59 - DNA, 21 bp, Artificial Sequence
  Description of Artificial Sequence Synthetic oligonucleotide
aactgacaataaccagaatgg

SEQ ID NO: 60 - DNA, 21 bp, Artificial Sequence
  Description of Artificial Sequence Synthetic oligonucleotide
aaattggctactaccgaagag

(19) United States
(12) Patent Application Publication
(10) Pub. No.: US 2007/0203082 A1
(43) Pub. Date: Aug. 30, 2007

(54) RNAI Agents For Anti-SARS Coronavirus Therapy

(75) Inventors: Quinn T. Tang, Gaithersburg, MD (US); Patrick Y. Lu, Rockville, MD (US); Frank Y. Xie, Germantown, MD (US); Yija Liu, Gaithersburg, MD (US); Jun Xu, Germantown, MD (US); Martin C. Woodle, Bethesda, MD (US)

Correspondence Address: PROSKAUER ROSE LLP, 1001 PENNSYLVANIA AVE, N.W.,, SUITE 400 SOUTH, WASHINGTON, DC, 20004, US

(73) Assignee: Intradigm Corporation, Rockville, MD (US)

(21) Appl. No.: 10/554,442

(22) PCT Filed: Apr. 26, 2004
(86) PCT No.: PCT/US04/12730

§ 371 Date: Dec. 12, 2006

Related U.S. Application Data
(60) Provisional application No. 60/465,216, filed on Apr. 25, 2003.

(51) Int. Cl. A61K 48/00; C12Q 1/70; C12Q 1/68; C07H 21/02

(52) U.S. Cl. 514/44; 435/5; 435/6; 536/23.1

(57) ABSTRACT

The present invention provides compositions and methods that are useful for the treatment of severe acute respiratory syndrome (SARS). More specifically, nucleic acid agents such as siRNA molecules and their analogues that target respiratory infections including SARS coronavirus and their methods of use are described, for clinical treatments of SARS, respiratory viral infections, for prevention and treatment of respiratory infections as needed for bio-defense, for treatment of respiratory diseases, and for discovery of therapeutic targets for respiratory diseases and infections.

This application claims priority to provisional Application No. 60/465,216, filed Apr. 25, 2003, the contents of which are hereby incorporated by reference in their entirety.

### FIELD OF THE INVENTION

The present invention provides compositions and methods that are useful for the treatment of severe acute respiratory syndrome (SARS). More specifically, nucleic acid agents such as siRNA molecules and their analogues that target respiratory infections including SARS coronavirus and their methods of use are described, for clinical treatments of SARS, respiratory viral infections, for prevention and treatment of respiratory infections as needed for bio-defense, for treatment of respiratory diseases, and for discovery of therapeutic targets for respiratory diseases and infections. The invention provides treatments and methods for human pulmonary diseases including genetic diseases, infectious diseases, pathological conditions, and autoimmune diseases. The invention also provides for siRNA agents and methods of delivery to inhibit expression of genes in animal disease models, such as mouse or monkey, as a means to discover and validate drug target function.

### BACKGROUND OF THE INVENTION

A new disease called severe acute respiratory syndrome (SARS) has recently been reported in Asia, North America, and Europe [1]. As of May 15, 2003 about 7628 cases of SARS had been reported, and 587 death worldwide. In general, SARS begins with a fever greater than 100.4° F. (>38.0° C.). Other symptoms may include headache, an overall feeling of discomfort, and body aches. Some people also experience mild respiratory symptoms. After 2 to 7 days, SARS patients may develop a dry cough and have trouble breathing. Most cases of SARS have involved people who cared for or lived with someone with SARS, or had direct contact with infectious material (for example, respiratory secretions) from a person who has SARS. Potential ways in which SARS can be spread include touching the skin of other people or objects that are contaminated with infectious droplets and then touching your eye(s), nose, or mouth. Because the etiology of these illnesses has not yet been determined, no specific treatment recommendations can be made at this time. Empiric therapy should include coverage for organisms associated with any community-acquired pneumonia of unclear etiology, including agents with activity against both typical and atypical respiratory pathogens. Treatment choices may be influenced by severity of the illness. Infectious disease consultation is recommended.

Similar to major challenges for respiratory infections, a number of pulmonary and respiratory diseases are not adequately treated including asthma and COPD. These and other respiratory diseases require better inhibitors of biochemical pathways associated with the disease. The present invention addresses the limitations in current treatments for respiratory and pulmonary disease using siRNA designed to inhibit selectively genes in the disease pathway and delivered in a manner as provided for by the invention.

### SUMMARY OF INVENTION

The present invention provides novel RNA interference (RNAi) agents and delivery methods for the inhibition of SARS-coronavirus (SARS-CoV) activity or other virus. The invention provides inhibition of viral production of key proteins required for replication, infection, and other functions critical to the virus lifecycle. The invention also provides disruption of the viral genome RNA directly. The invention provides:

Sequences of RNAi agent, small interfering RNA (siRNA), that can be chemically synthesized or vector expressed, in vitro transcribed and vector expressed shRNA; siRNA, miRNA and other types of siRNA molecules, having potent antiviral activity in mammalian cells and animals;

Agents useful for siRNA-mediated gene inhibition in mammalian cells and animal airways and lung tissues;

Agents useful for efficient delivery of siRNA into the airways of animal model;

Mechanism of action of SARS-CoV specific siRNA duplexes for inhibition of the viral infection and replication in mammals;

Target sequences coding for key proteins required for corona virus replication and infection;

Target sequences for siRNA-mediated disruption of corona virus viral RNA genome in coding and non-coding regions;

Routes and methods of delivery for nucleic acid agents and analogues for mammals;

Methods and reagents for RNA template-specific RNA based RT-PCR for detection of any portion of the viral RNA genome, for applications of diagnosis and prognosis; and

Methods for using nucleic acid agents and analogues to treat pulmonary diseases and infections.

More specifically, the invention provides an isolated double stranded RNA molecule containing a first strand having a ribonucleotide sequence which corresponds to a nucleotide sequence of a SARS virus and a second strand having a ribonucleotide sequence which is complementary to the nucleotide sequence of the SARS virus, where the double-stranded molecule inhibits expression of the nucleotide sequence of the SARS virus.

The first and second strands may be separate complementary strands, or may be contained in a single molecule, where the single molecule contains a loop structure. The nucleotide sequence of a SARS virus may be an nsp1 sequence, an nsp9 sequence or a spike sequence, for example.

The first strand may contain a sequence selected from the group consisting of AACCTTTGGAGAAGATACTGT, AATCACATTTGAGCTTGATGA, AAGTTGCTGGTTTTGCAAAGT, AAGGATGAGGAAGGCAATTTA, AAGCTCCTAATTACACTCAAC, and AATGTTACAGGGTTTCATACT.

the invention also provides a method of detecting a SARS virus in a sample, by (a) contacting RNA obtained from the sample with a gene specific primer containing a 3′ region that is complementary to a SARS sequence and a 5′ sequence that is not complementary to a SARS sequence and synthesizing a first strand cDNA molecule by reverse transcription followed by (b) amplifying the first strand cDNA in a PCR using a pair of primers, where the first primer is complementary to the 5′ region of the gene specific primer and where the second primer contains a sequence in the SARS genome that is upstream of the region recognized by the 3′ region of the gene specific primer, and (c) detecting the product of the PCR. The gene specific primer may be complementary to a SARS nps1, nps9 or spike sequence, for example. The gene specific primer may contain a sequence selected from the group consisting of GAA CAT CGA TGA CAA GCT TAG GTA TCG ATA gac aac ctg ctc ata aa, GAA CAT CGA TGA CAA GCT TAG GTA TCG ATA gag gat ggg cat cag ca, and GAA CAT CGA TGA CAA GCT TAG GTA TCG ATA gtg tta aaa cca gaa gg. The first primer may contain the sequence GAACATCGATGACAAGCTTAGGTATCGATA. The second primer may contain a sequence selected from the group consisting of

GGG AAG TTC AAG GTT ACA AGA ATG TGA GAA,
CGG TGT AAG TGC AGC CCG TCT TAC ACC GTG,
and
CCT TGA CCG GTG CAC CAC TTT TGA TGA TGT.

The invention further provides a method of treating or preventing a coronavirus infection in a subject, such as a SARS virus infection, by administering to the subject an effective amount of a composition containing an isolated double stranded RNA molecule, where the RNA molecule contains a first strand containing a ribonucleotide sequence which corresponds to a nucleotide sequence of a coronavirus and a second strand containing a ribonucleotide sequence which is complementary to the nucleotide sequence of the coronavirus, where the double-stranded molecule inhibits expression of the nucleotide sequence of the coronavirus. The first and second strands may be separate complementary strands, or may be contained in a single molecule, where the single molecule contains a loop structure. The nucleotide sequence from the SARS virus may be an nsp1 sequence, an nsp9 sequence or a spike sequence, for example. The first strand may contain a sequence selected from the group consisting of AACCTTTGGAGAAGATACTGT, AATCACATTTGAGCTTGATGA, AAGTTGCTGGTTTTGCAAAGT, AAGGATGAGGAAGGCAATTTA, AAGCTCCTAATTACACTCAAC, and AATGTTACAGGGTTTCATACT. The double stranded RNA molecule may contain a sequence selected from the group consisting of SC2, SC5, SC14 and SC15.

In the above methods of treatment or prevention, the double stranded RNA molecule may be delivered into the airway of the subject, for example by intranasal delivery or by delivery into the trachea. The composition may contain the double stranded RNA molecule in a carrier containing an aqueous glucose solution free of RNAse, such as a 5% glucose solution. The dosage of the double stranded RNA molecule may be 1-100 mg per kg of body weight of the subject. The composition may also be delivered as an aqueous RNA-free solution, in an aerosol or in a powder.

The invention also provides a method of treating a respiratory disease in a subject, by administering to the airway of the subject a double stranded RNA molecule containing a first strand containing a ribonucleotide sequence which corresponds to a nucleotide sequence of a gene implicated in the disease and a second strand containing a ribonucleotide sequence which is complementary to the nucleotide sequence of the nucleotide sequence of the gene, where the gene implicated in the disease exhibits undesirably high levels of gene expression in the disease, and where the double-stranded molecule inhibits expression of the nucleotide sequence of the gene implicated in the disease. The gene implicated in the disease may be a gene of a pathogenic organism, such as a bacterium, a virus or a fungus. The disease also may, for example, autoimmune inflammation or lung cancer.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows the Genomic Organization of SARS Coronavirua CUHK-WIUrbani Genomic sequence of the SARS coronavirus CUHK-WI strain (AY278554.1), which is 29206 bps long. The sizes of the genes are drawn about to scale. Structural proteins are shown as solid box. L, leader sequence; “p65?” indicates putative MHVp65-like protein; number 1-13 show non-structural (nsp) proteins, where nsp-8 is missing in published sequence data. S, spike protein; M, membrane glycoprotein; U, unknown proteins. Arrows show non-structural polyproteins. Black bars show the position of the siRNA-targeted sequences.

FIG. 2 shows the Genomic Organization of SARS Coronavirua Urbani strain (AY278741.1), which is 29727 bps long. The sizes of the genes are drawn about to scale. Structural proteins are shown as solid box. S, spike proteins; E, envelope protein; M, membrane glyoprotein; N, nucleocapsid phosphoprotein. Arrows show non-structural polyproteins. Numbered black bars show the position of the siRNA-targeted sequences.

FIG. 3 shows the location of siRNA targets on different SARS coronavirus isolates Target sequences as designed based upon SARS coronavirus CUHK-WI were used to find it's specificity for different SARS coronavirus isolates. The “mis-match” of the fifth and sixth target sequences (Spike-1 & 2) with GZ-01 isolate was simply because the incomplete sequence data of GZ01 isolate as submitted; and the mis-match of the third target sequence (nsp9-A) on HKU39849 was because there is one base pair missing in HKU39849 sequence at position 13496 nt, which was not found in genomic sequence of other isolates.

FIG. 4 shows nucleic acid delivery to the pulmonary system. Airway delivery is very effective through multiple routes. Aerosol, intranasal installation and oral-tracheal delivery are non-invasive routes for delivery of RNAi molecules.

FIG. 5 shows inhibition of luciferase expression by siRNA in the lung. Luciferase plasmid together with siRNA specific for either GFP or luciferase were oraltracheally into mice, using either 5% glucose or Infasurf. Luciferase activity was measured 16 hrs later in lung homogenates.

FIG. 6 shows the distribution of fluorescence-labeled siRNA in the respiratory tract of mice using the nostril delivery route Thirty ug of fluorescein-labeled siRNA duplex in 50 ul nostril delivery solution (5% glucose and 12 ug/ul infasurf) was delivered into the respiratory tract through the nostril delivery route. Four hours post delivery, the animal was sacrificed and the respiratory trachea and lung were isolated. Examination of tissues under fluorescence microscopy revealed massive distribution of siRNA in the respiratory tract and lung, even after washing tissues with PBS to remove siRNA non-specifically attached to cell surface.

FIG. 7 shows the distribution of fluorescence-labeled siRNA in the respiratory tract of mice using Oral-tracheal delivery route Thirty ug of fluorescein-labeled siRNA duplex in 50 ul oral-tracheal delivery solution (5% glucose and 12 ug/ul infasurf) was delivered into the respiratory tract through the nostril delivery route. Four hours post delivery, the animal was sacrificed and the respiratory trachea and lung were isolated. Examination of tissues under fluorescence microscopy revealed massive distribution of siRNA in the respiratory trachea and lung, even after washing tissues with PBS to remove siRNA non-specifically attached to cell surface.

FIG. 8 shows the locations of 48 siRNA targeting sequences within the SARS-CoV genome. The entire genome, about 29.7 kb, consists of 14 ORFs coding at 5′ end for both the replicase and transcriptase, and at 3′ end for the structural and accessory proteins. 16 duplexes target the ORF1a and ORF1b regions, while 32 duplexes target regions from ORF2 to ORF9. The regions coding for the Spike protein, membrane glycoprotein, envelope protein and ORF3 were heavily targeted with 6 or 7 duplexes each. The bold bars indicate the locations of each siRNA-targeted sequence. The arrows point out the sequences that resulted in strong anti-SARS-CoV activities.

FIG. 9 shows the 48 siRNA molecules used for cell culture transfection to test their anti-SARS-CoV activities.

FIG. 10 shows the antiviral effects of siRNA in FRhK4 cells. A, B and C illustrate the CPE of the cells in response to SARS-CoV infection. When healthy cells (A) were infected by the virus, marked CPE was observed (B), versus cells were first transfected with the siRNA duplex then infected by the virus (C) where no visible CPE occurred.

FIG. 11 shows electron microscopy of SARS-CoV, indicated by arrows within the infected cell (D), versus no virus visible in the cell protected by the transfection of siRNA first and then infected by the virus (E).

FIG. 12 shows the prophylactic effects of the selected siRNA duplexes detected with relative viral genome copies. Four siRNAs, SC2, SC5, SC14 and SC15, selected from the CPE screening of all 48 duplexes, were tested for their potencies as the prophylactic agents in FRhK-4 cells. Detection with real-time quantitative RT-PCR revealed that these siRNA duplexes were able to significantly (p<0.01) reduce viral replication.

FIG. 13 shows the prophylactic effects of the selected siRNA duplexes detected with relative viral yield (TCID₅₀) in the medium. The siRNA pre-treated groups were significantly (p<0.01) reduced comparing to control groups without pre-treatment.

FIG. 14 shows the duration of the siRNA-mediated prophylactic effect. FRhK-4 cells were infected at 4, 8, 16, 24, 48, 60, and 72 hours post transfection of SC5 siRNA. 36 hours later, and the viral titers were measured for evaluation of the prophylactic effect of siRNA against SARS-CoV infection at different time points. The black bar indicates the relative viral genome copy of sample not pre-treated with the siRNAs, versus the white bar for pre-treated samples. Three replicates were tested for each sample and standard deviations are illustrated.

FIG. 15 shows the therapeutic effects of selected siRNA duplexes detected with viral genome copy numbers in the cell culture. FRhK-4 cells were infected with SARS-CoV followed by transfection of SC2, SC5, SC14 and SC15 siRNA duplexes. Measurements of the therapeutic effects were conducted at 36 hours post transfection. Three replicates were tested for each sample and the standard deviations are illustrated.

FIG. 16 shows the therapeutic effects of selected siRNA duplexes detected with viral titration (TCID₅₀). Three replicates were tested for each sample and the standard deviations are illustrated.

FIG. 17 shows the therapeutic effects of combined siRNA duplexes. Relative viral genome copies were measured after FRhK4 cells were infected by SARS-CoV followed by the transfection by the active siRNA duplexes with various combinations. At 36 hours post transfection, cells and culture medium were collected for Q-RT-PCR and viral titer. Significant anti-viral therapeutic effects were observed with infected cells treated with the combined siRNA duplexes. Three replicates were tested for each sample and the standard deviations are illustrated.

FIG. 18 shows the prophylactic effects of various siRNA combinations on relative viral genome copy numbers. Seven combinations with the four selected siRNA duplexes were transfected into FRhK-4 cells 8 hours before the SARS-CoV infection. Samples were collected 24 hours post infection for Q-RT-PCR.

FIG. 19 shows a time-course of the protective effect of the SC2 and SC5 siRNA combination. The black bar indicates the relative viral genome copy of sample not pre-treated with the siRNAs, versus the white bar for pre-treated samples. Three replicates were tested for each sample and the standard deviations are illustrated.

FIG. 20 shows the mammalian expression vector, pCI-Luc-SC, constructed with CMV driven Luciferase fused with SARS-CoV sequences including SC2 and SC5. When the SC2 and SC5 siRNA duplexes and this vector were co-transfected into 293 cells, Luciferase expression levels were significantly down-regulated.

FIG. 21 shows the effect 24 hours after pCI-Luc-SC plasmid was co-delivered with SC2 and SC5 siRNA duplexes into mouse lung through intratracheal administration. siRNA-mediated sequence specific knockdown is indicated by inhibition of Luciferase expression in the lung.

FIG. 22 shows pathohistological data of a non-human primate study using the combined siRNA duplexes to inhibition SARS-CoV infection in the lungs. 5 groups of testing animal with 4 monkeys per group were treated by either SARS-CoV infection alone or co-delivered at different time points of SARS-CoV and the siRNA duplexes through intranasal delivery of 0.5 ml of saline solution. Group I was treated with SC2 and SC5 siRNA (30 mg per dose) combination before SARS-CoV infection. Group II was treated with SC2-SC5 siRNA and SARS-CoV co-administration (30 mg per dose) followed by two additional doses. Group III was treated with SARS-CoV virus first and then 3 times with repeated delivery of the SC2-SC5 siRNA combination. Group IV was treated with a control siRNA with the same dosage following SARS-CoV infection. Group V was infected only by SARS-CoV. The Monkeys were sacrificed and the lung tissues were collected for pathohistological analysis. Group I and Group II demonstrated much less pathological changes than those of the Group IV and V.

FIG. 23 shows pathohistological staining of monkey lung indicating pathological changes.

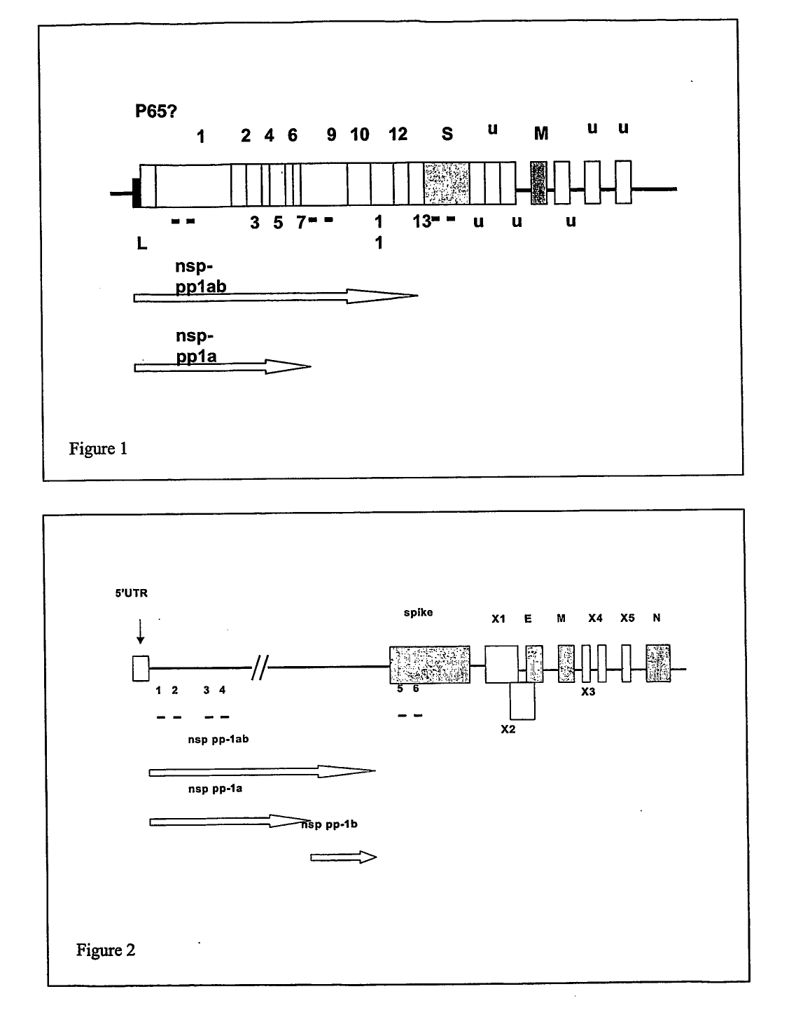

### DETAILED DESCRIPTION OF THE INVENTION

The present invention provides compositions and methods for treating coronavirus infections in mammals, especially in primates and humans, by inhibiting coronavirus gene expression using siRNA molecules delivered in vivo.

More specifically, the invention provides for inhibition of genes or genomic material in pulmonary tissues. By inhibiting genes or genomes of virus, treatments or preventative therapies for infectious diseases are provided. The invention provides for short, double stranded RNA oligonucleotides, or siRNA, that inhibit expression of genes with a matching sequence or inhibit RNA virus genomes. The invention also provides nucleic acid (including RNA or DNA) therapeutic agents. The invention also provides methods of delivery to pulmonary tissues. In one embodiment, the invention provides inhibitors of corona virus and in particular SARS corona virus. These inhibitors of respiratory infections, including respiratory virus infections, can be used as therapeutic treatments and they can be used as preventative treatments.

By inhibiting mammalian genes, treatments of diseases are provided. Many genes have been identified for a role initiating, maintaining, or exacerbating pulmonary diseases. For example, COPD is characterized by inflammation and degradation of pulmonary tissues and many genes have been identified with these destructive processes of COPD including numerous cytokines and numerous proteases. Similarly, asthma is characterized by unwanted constriction of airways and many genes have been identified with this process including ion channels. Thus the invention provides for inhibition of mammalian genes that initiate these processes, that maintain these processes, and that exacerbate these processes. The inhibitors provided by the invention provide therapeutic treatments for pulmonary diseases.

The invention provides for inhibitors for respiratory infections that result from natural or engineered changes in infectious agents. Such natural or engineered changes in infectious agents that result in new infectious agents cause new respiratory infections. These new infections require new therapeutics. The invention provides therapeutic methods to inhibit such new infectious agents simply by obtaining the genome of the new agent and identifying siRNA targeting unique sequences.

SARS

Scientists in many laboratories in Asia, Europe and North America have been working on the cause of SARS around the clock. A previously unrecognized coronavirus in patients with SARS has been isolated, sequenced and tested in a monkey model [24]. This new coronavirus, which is the leading candidate for causing SARS, has been named SARS coronavirus by the World Health Organization. However, other infectious agents are still under investigation as potential causes of SARS. Currently, there are multiple genome sequences of the SARS-CoV being reported by groups in U.S., Canada, Hong Kong, Netherlands and elsewhere [5]. The sequence information provides critical knowledge bases for designing diagnostic reagents employing either RT-PCR or ELISA. More importantly, based on these sequences, we designed siRNA duplexes to knock down several important viral proteins, theoretically all of them are able to disrupt the positive strained viral genome, thus to inhibit the replication process of SARS-CoV. This success in generating such siRNA duplexes permits development of siRNA-based therapeutics to be delivered into patient airways for both prevention and therapy of SARS. SARS-CoV is a sense and single stranded RNA, can cause one of the most prevalent infections in humans. The virulence of SARS-CoV results from i) its easy spread by aerosol and other person-to-person contacts, ii) its ability to escape from protective immunity by frequent changes in viral antigens (a characteristic of almost all RNA viruses), and iii) the sharp emergence of new virulent strains of the virus. The threat of the possible new strain of SARS-CoV is severe because, despite intensive efforts, no effective therapy or vaccine is yet available for prevention and treatment of the SARS-CoV infection, and there are so many epidemiological, etiologic details of this disease left unknown.

RNA Interference (RNAi) Inhibits SARS-CoV Infection And Replication.

RNAi is a process by which double-stranded RNA directs sequence-specific degradation of messenger RNAs in animal and plant cells [6-8]. One form of RNAi, small interfering RNA (siRNA, 21 nt in length) duplexes, has been proven effective in blocking viral infection and replication in vitro and in vivo [9-12]. This approach is particularly useful for a group of RNA viruses, HUV, HCV and influenza, etc., resulting in significant inhibitions of viral infection in various mammalian cell systems and animal model systems [13-23].

RNAi appears to be ideal for interfering with SARS CoV infection. First, SARS CoV is a single stranded RNA virus, without any DNA intermediates during its life cycle. Besides mRNA, vRNA and cRNA are potential targets for siRNA-mediated degradation. Second, SARS CoV genomic RNA encodes multiple proteins. Each protein either is an integral part of the viral structure or plays a critical role during the virus life cycle. Interfering with the production of any single protein is likely to have severe consequences on viral replication and production. Thus, the virus presents multiple siRNA targets, and combinations of siRNAs against different viral targets may be used simultaneously. The use of two or more siRNAs simultaneously may be required to prevent the emergence of resistant virus, analogous to the use of drug “cocktails” for HIV-treatment. Third, SARS-CoV infects epithelial cells in the upper respiratory tract and the lungs in humans. Thus, siRNAs can be administered conveniently via intranasal or pulmonary routes, which, in turn, may result in a much higher local siRNA concentration than that achieved by systemic injection. Considering that the number of virions probably is small at the beginning of a natural infection, sufficient amounts of siRNA may be taken up by epithelial cells in the upper airways and the lungs to inhibit virus replication or production, thus potentially achieving preventive or therapeutic effects.

Multiple siRNA duplexes are described herein that target sequences encoding key proteins required for SARS-CoV infection and replication in humans. As a result of its single stranded RNA genome structure, SARS-CoV can be directly killed by siRNA-mediated RNA degradation. To use these siRNA duplexes for prophylaxis and therapy of SARS-CoV infection in humans, the siRNAs must be delivered into epithelial cells in the upper airway and the lung, where the virus infection normally occurs.

The success of pre-clinical study of the siRNA-based therapeutics for anti-SARS-CoV efficacy depends on:

1. anti-viral activity of the siRNA duplexes;

2. delivery efficiency of siRNA duplex into animal airways and

3. tolerable toxicity in clinical relevant animal models.

siRNA duplexes were designed that potently inhibit SARS coronavirus production in cultured cells and animal models. To use these RNAi duplexes for prophylaxis and therapy of SARS-CoV infection in humans, the siRNAs must be delivered into epithelial cells in the upper airway and the lung, where the virus infection normally occurs.

Identification of Potent siRNA Molecules In Vitro

Methods and Materials

SARS Coronavirus Strain Selection

SARS-CoV strain HKU-66078 isolate (AY304494) was isolated by infection of fetal rhesus kidney (FRhK-4) cells with the nasopharyngeal aspirate (NPA) of a patient who suffered from SARS in March 2003 in Hong Kong [24] using procedure described previously [1]. Serial passages of HKU-66078 strain in FRhK4 cells consistently yielded cytopathic effect (CPE) with a titer of 10⁷ TCID₅₀/ml. Parcel-length sequencing and phylogenetic analysis showed that this strain closely resembles the reported strains, TOR2 (AY274119), FRA (AY291315 and AY310120) and CUHK-WI (AY278554). This strain was chosen due to its high infectious and virulence property that resulted in CPE faster than other strains (unpublished data).

Cell Culture, Transfection and Viral Infection

FRhk-4 cells were cultured in 96-w plates in MEM medium with 10% of FCS. For viral infection, cells were washed twice with PBS, inoculated with 3 PFU/cells of the virus and incubated for one hour in MEM without FCS. The cells were then washed twice with MEM and cultured for 24 hours or longer in MEM medium containing 10% FCS at 37° C. in CO2 incubator. The CPE appeared about 20 hours post infection, and spread quickly to the entire cell monolayer within another 28 hours. For prophylactic study, the FRhK4 cells in 96-well plate at 90-95% confluency were transfected with siRNA duplex at 0.3 μg/well mixing with 0.5 μl of Lipofectamine 2000 (Invitrogen, Carlsbad, Calif., USA) following manufacture's procedure. Eight hours post transfection, cells were infected with SARS-CoV at 3 PFU/cell. For therapeutic study, the FRhK4 cells in 96-well plate at 80% confluency were infected with SARS-CoV at 3 PFU/cell. At 1 hour post infection, the cells were transfected with siRNA duplex at 0.3 μg/well mixing with 0.5 μl of Lipofectamine 2000 following manufacture's procedure. Four hours post the transfection, cells were washed and cultured in MEM medium with 10% of FCS.

Design and Synthesis of siRNA

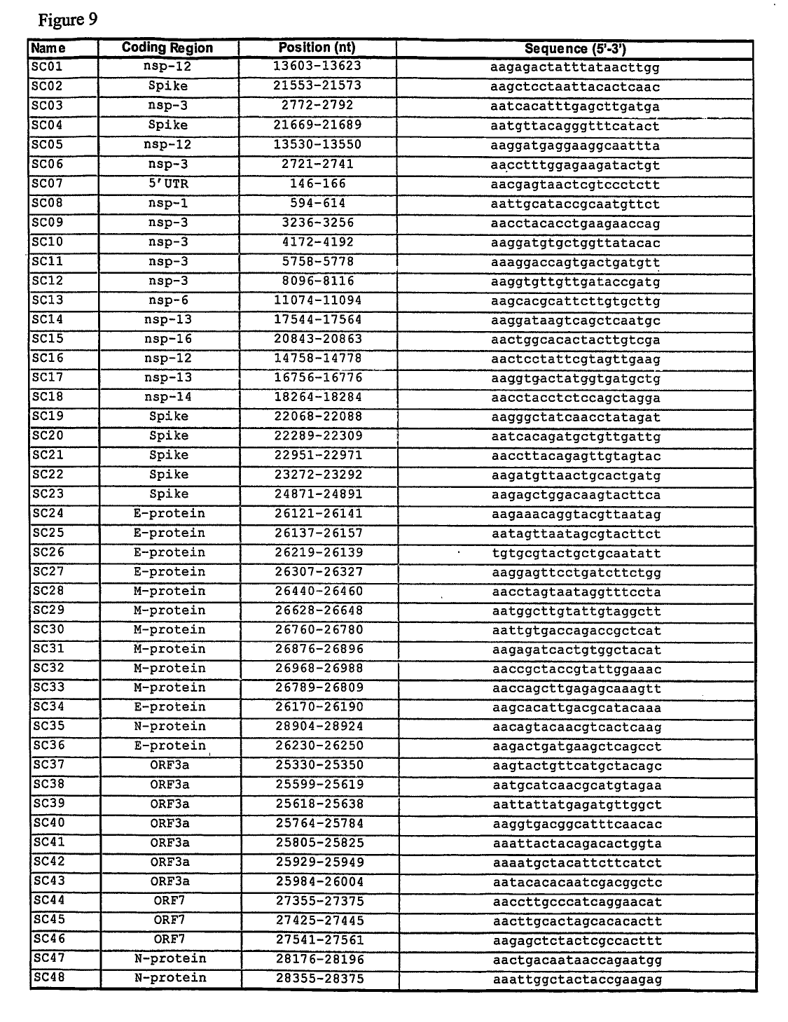

SARS-CoV genome sequences based on TOR-2 (AY274119), CUHK-WI (AY278554) and HKU-66078 (AY304494) were used as the templates for designing siRNA target sequences. All siRNA duplexes were double-stranded RNA of 21 nucleotides (nt) containing dTdT overhung at both 3′ ends according to the rules suggested by Elbashir et al. [25]. The target sequences were subjected to a BLAST search against GenBank to ensure that they are unique to only SARS-CoV genome sequences. Additional 40 siRNA duplexes were also designed (FIG. 9) and synthesized by Qiagen (Germantown, Md.).

Electron Microscopy

FRhk-4 cells with or without SARS-CoV infection were harvested and fixed in 2.5% glutaraldehyde (Electron Microscopy Sciences, Washington, USA) for 4 hours and post-fixed in 1% osmium tetroxide for 1 hour. The cells were then transferred to a 1.5 ml tube and centrifuged at 1,000 rpm for 10 minutes. Upon removal of the supernatant, a liquidized 2% agarose (Sigma, St. Louis, USA) solution at 55-60° C. was added to the cell pellet. After solidification of the gel, approximately 1 mm³ cubes containing cell pellet were prepared and dehydrated in graded ethanol. The cubes were embedded in epoxy resin (Polysciences, Warrington, USA). Ultra-thin sections with 70 nm thickness were prepared and stained with uranyl acetate (Electron Microscopy Sciences, Washington, USA) and lead citrate (Leica Microsystem, Vienna, Austria). The sections were examined under a Philips EM208S electron microscope at 80 kV. The images were marked with 200 nm in length.

Virus Titration and Real-Time Quantitative RT-PCR

The released virus in the culture medium was determined by titration of viral yield in the culture supernatant using CPE-based TCID50 test The culture supernatant was serially diluted at 10 fold with MEM and inoculated to the FRhK-4 cells in 96 well plate. The results were evaluated after 3 days of culture. Intracellular copy numbers of viral genome RNA were quantified using a real-time quantitative RT-PCR (Q-RT-PCR). The cells were washed twice with PBS, and total RNA was extracted from the cells using a QIAamp RNA Isolation Kit (Roche Molecular Biochemicals). First strand cDNA was synthesized using RNA H⁺ Reverse Transcriptase (Invitrogen) and random primers. Two micro liters of reverse transcription products from each reaction was used for PCR. The forward primer (5′-GCATGAAATTGCCTGGTTCAC-3′, at a final concentration of 900 nM), reverse primer (5′-GCATTCCCCTTTGAAAGTGTC-3′, at a final concentration of 900 nM) and fluorescence probe (FAMAGCTACGAGCACCAGACACCCTTCGAAA-TRMA, at a final concentration 250 nM) were mixed with Master Mix and subjected to real-time PCR using ABI7900 Sequence Detection System (ABI, Foster City, Calif., USA). The conditions for running PCR were: 50° C. for 5 minutes, 95° C. for 10 minutes and 40 cycles of 95° C. for 15 seconds and 61° C. for 1 minute. All measurements were conducted 3 times for statistical analysis.

Results

Selection of the Most Potent siRNA Duplexes

Though siRNA duplexes in general are able to knockdown complementary RNA sequences, it is known that siRNAs that target different regions of the same gene vary markedly in their silencing effectiveness. While the rules that govern efficient siRNA-directed gene silencing remain undefined, the base composition of the siRNA sequence is probably not the only determinant of how efficiently it will knockdown a target gene. The strategy taken in this study was a permutation of focusing regions coding for certain key proteins for SARS-CoV infection and replication and meanwhile covering regions throughout the entire viral genome RNA, to ensure that the potent siRNA duplexes for inhibition of SARS-CoV can be identified. Forty-eight sequences with 21 nt each were selected as targets for siRNA-mediated inhibition of SARS-CoV infection and replication. The locations of each siRNA-targeted sequences within the genome RNA are illustrated in FIG. 8 according to the SARS-CoV genome organization described recently [27-29] and the details of each siRNA targeted sequence are listed in FIG. 9.

Sequence analysis revealed the organization of the 29,740 base (FRA isolate, AY310120) genome of the SARS-CoV [27-29]. Nucleotides 1-72 contain a predicted RNA leader sequence preceding an untranslated region (UTR) spanning 192 nucleotides [26]. SARS-CoV genome expression starts with the translation of two large replicase open reading frames, ORF1a and ORF1b, both coding for polyproteins that are processed into a group of poorly characterized replicative enzymes. These replicase subunits are speculated to form a viral replication complex responsible for the synthesis and replication of viral RNA in the host cells [29]. Among them the papain-like cysteine protease (PLpro) coded by nsp-3 region is important for the maturation of viral proteins, and the RNA-dependent RNA polymerase coded by nsp-12 region plays a critical role in catalyzing the synthesis of viral RNAs. Spike protein coded by the ORF2 and located on the surface of virion, is responsible for tropism, receptor recognition, cell adsorption, and induction of neutralizing antibody as well. Initially, an understanding of the functional roles of the viral sequence led the rational design of 8 siRNA duplexes (SC1-SC8) targeting the leader sequence, nsp-3, nsp-12 and Spike coding regions. The siRNA duplexes were transfected into FRhk-4 cells that were lately infected with SARS-CoV. The cytopathic effect (CPE) of the treated cells was evaluated 36 hours post infection as the indication for siRNA-mediated protection from the viral infection. One nsp-12 specific siRNA, SC5, and one Spike protein specific siRNA, SC2, demonstrated significant reduction of CPE (>80%), while the other 6 duplexes showed only moderate (50%-70%) or minimum reduction (<30%) of CPE.

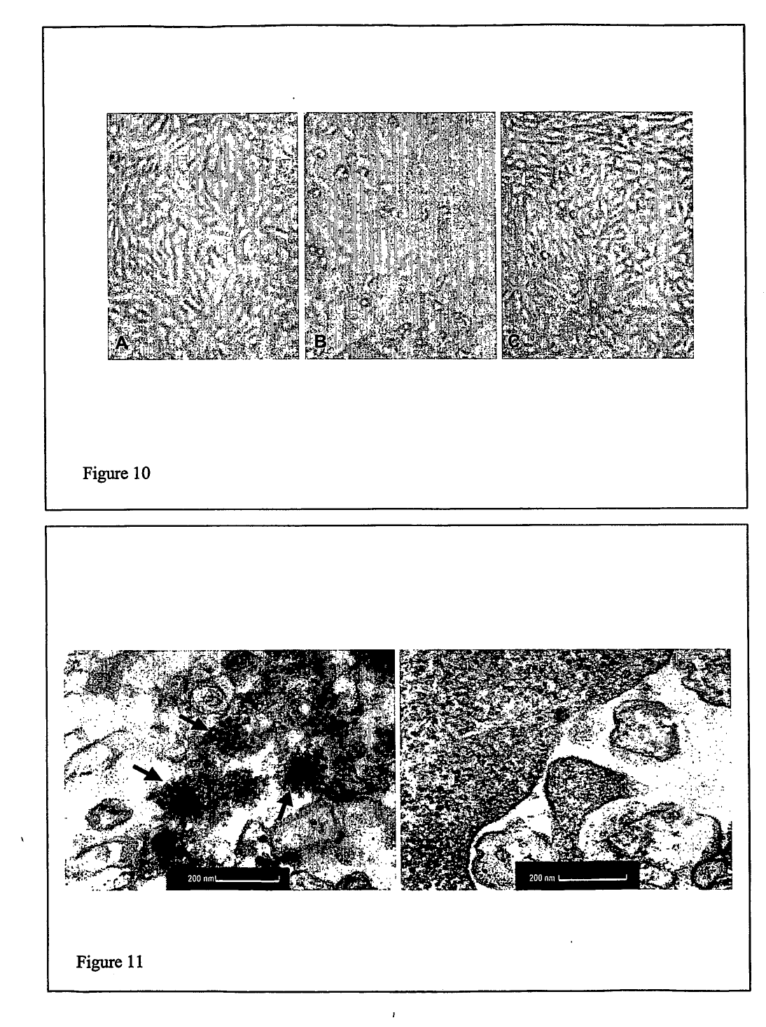

This initial success prompted a genome-wide screening with additional 40 siRNA targeting various regions throughout the entire SARS-CoV genome RNA (FIG. 8), using the same CPE based procedures. Surprisingly, only two additional siRNA duplexes, SC14 targeting the nsp-13 region and SC15 targeting the nsp-16 region, showed a similar potency in reducing CPE as observed with SC2 and SC5. FRhK-4 cells transfected with SC14 siRNA into prior to infection with SARS-CoV, exhibited a profound prophylactic protection of the cells from CPE (FIG. 10). Synthetic siRNA duplexes have been demonstrated to be capable of degradation of the viral genomic RNA when cells are transfected with siRNA prior to HUV viral infection [30]. As siRNA operates in the cytoplasm, genomic viral RNAs that enter cells during infection have to encounter this initial defensive machinery.

The ability of siRNA to target incoming genomic viral RNA has implications for therapeutic use of siRNA in SARS-CoV infection treatment. The protection of FRhK-4 cells from the SARS-CoV infection was further illustrated through the electron microscopy images (FIG. 11). Nevertheless, only 4 out of 48 siRNA duplexes showed a significant reduction of CPE from by SARS-CoV infection. The GC contents of these four siRNA duplexes range from 38% to 48%. It appears that the position of the siRNA target sequences in the viral genomic has a direct impact on the efficiency of viral RNA disruption. More interestingly, all of these four most potent inhibitors, SC2, SC5, SC14 and SC15 targeted the middle of the viral genome sequence (nt 13500-21600). The fact that three of them directly targeted the ORF1b region strongly supported the notion that this coding region may play a critical role in the viral genome stability. Furthermore, the strong inhibitory effects of only 3 out of 7 siRNA duplexes targeting the ORF1b region showed the sequence preferences within the coding region.

There were no clear sequence pattern and motif character for better susceptibility of RNAi within these three duplexes being recognized. The exact mechanism of the position effect at a viral genome level observed in this study requires further investigation for complete understanding. SC2 siRNA, 1 out of 7 siRNA duplexes targeting Spike protein were able to significantly reduce SARS-CoV induced CPE. This further confirmed that the position effects of siRNA target sequence within each ORF may vary substantially [25].

Prophylactic Effects of the Selected siRNA Duplex

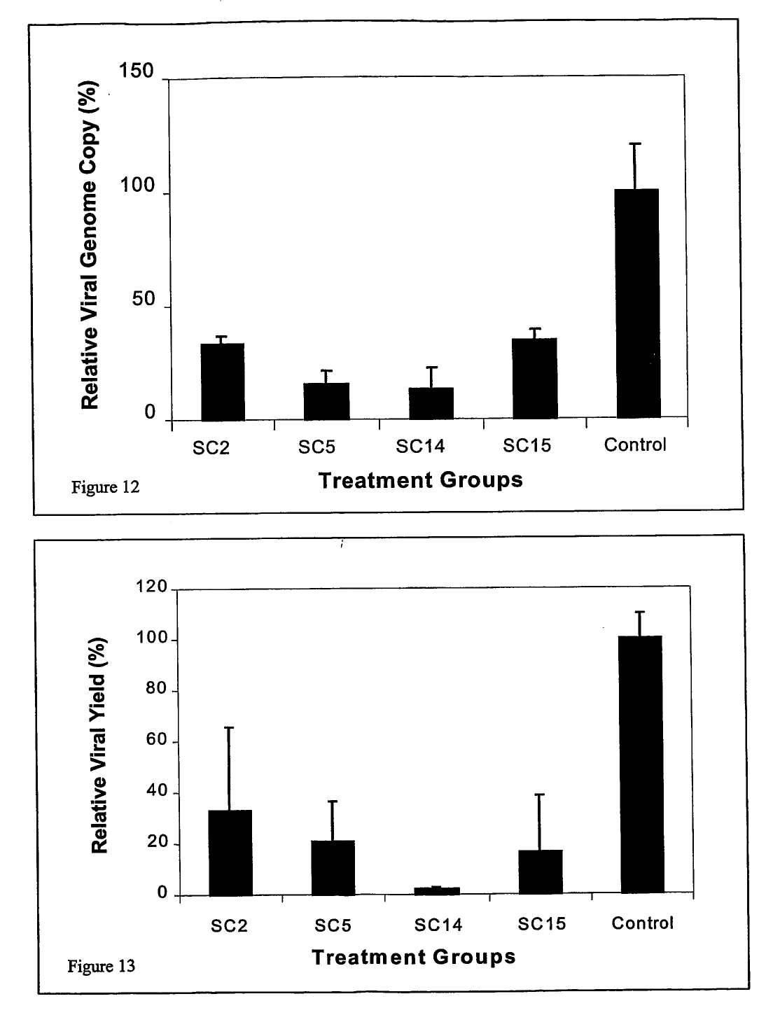

Studies of the HIV-1 [13-14] and respiratory syncytial virus (RSV) [30] have indicated that both viral genomic RNA and mRNAs are sensitive to preexisting siRNA within host cells. To investigate prophylactic effects of selected active siRNA targeting SARS CoV, FRhK-4 cells were transfected with the siRNA duplexes prior to the viral infection. The antiviral efficacy was evaluated by measuring the cytoplasmic viral genome copy number, using Q-RT-PCR, and titrating viral yield (TCID₅₀) in the culture media. FIG. 12 shows the reduction of SARS-CoV genome copy number with transfection of siRNA duplexes SC2, SC5, SC14, and SC15 into FRhK-4 cells 8 hours prior to the viral infection. The relative viral genome copy numbers were measured using a Q-RT-PCR from samples harvested 72 hours post infection.

FIG. 13 shows the inhibitory effect of the siRNA duplexes on SARS-CoV yield in the culture medium. Both measurements demonstrated that SC5, SC14 and SC15 siRNA duplexes were able to achieve the substantial inhibition of viral replication, while SC14 exhibited the greatest potency. The observed prophylactic inhibitory effects provided direct evidence that preexisting siRNAs in the host cells are able to prevent SARS-CoV infection and inhibit the viral replication.

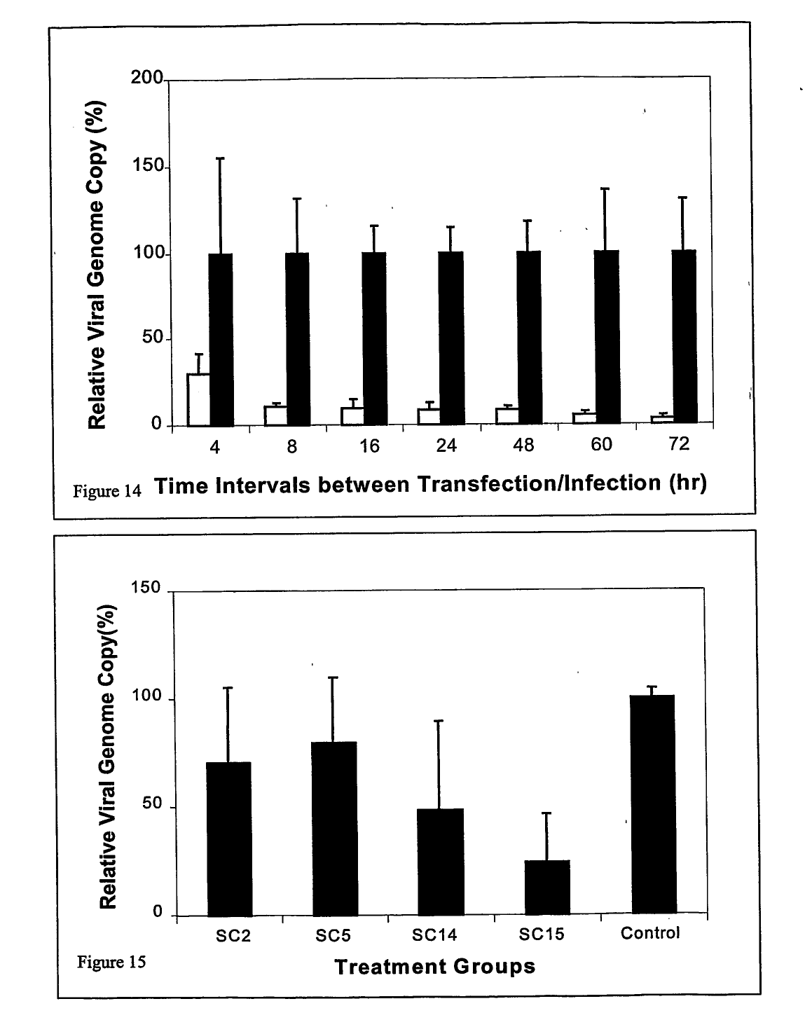

One question studied was how long this siRNA-mediated prophylactic effect can last after single transfection of the anti-SARS-CoV siRNAs. To address this, a time course study was conducted to define the duration of siRNA-mediated prophylactic activity. FRhK-4 cells were infected with same doses of SARS-CoV at time points of 4, 8, 16, 24, 48, 60 and 72 hours after the transfection of same dose of SC5 siRNA. siRNA-mediated anti-SARS-CoV prophylactic effect was maintained for up to 72 hours, the longest time period in the study (FIG. 14), even though there have been reports about relatively stable and long lasting siRNA-mediated silencing effects [25, 31]. During the time course, the viral genome copy numbers of pretreated groups remained low at all time points, comparing to a rapid increase of viral genome copy numbers 8 hours post infection in the absence of siRNA. This result indicated that the siRNA duplex remained stable and active in the FRhK-4 cells for at least 72 hours. This prolonged prophylactic effect suggests the potential use of siRNA as a preventative measure against SARS-CoV infection, such as administrating the siRNA to the health care professionals prior to their exposure to SARS patients, since the prophylactic siRNA is able to act promptly within hours and last for days. The prophylactic antiviral effect might also provoke a worthwhile investigation of the mechanism how preexisting siRNA agent can prevent viral infection of the host cells.

Therapeutic Effects of the Selected siRNA Duplex

At the early stage of viral replication, the RNAi machinery only has to deal with genomic RNA. However, after virus enters the cell, the replication activates and thousands of viral transcripts are generated de novo in the infected cells, and the degradation of the viral genomic RNA and mRNA become a far greater task for RNAi machinery. To evaluate the therapeutic effects of the selected siRNA duplexes, FRhK4 cells were transfected with the same dosage of the siRNAs used in the prophylactic study, one hour after the SARS-CoV infection.

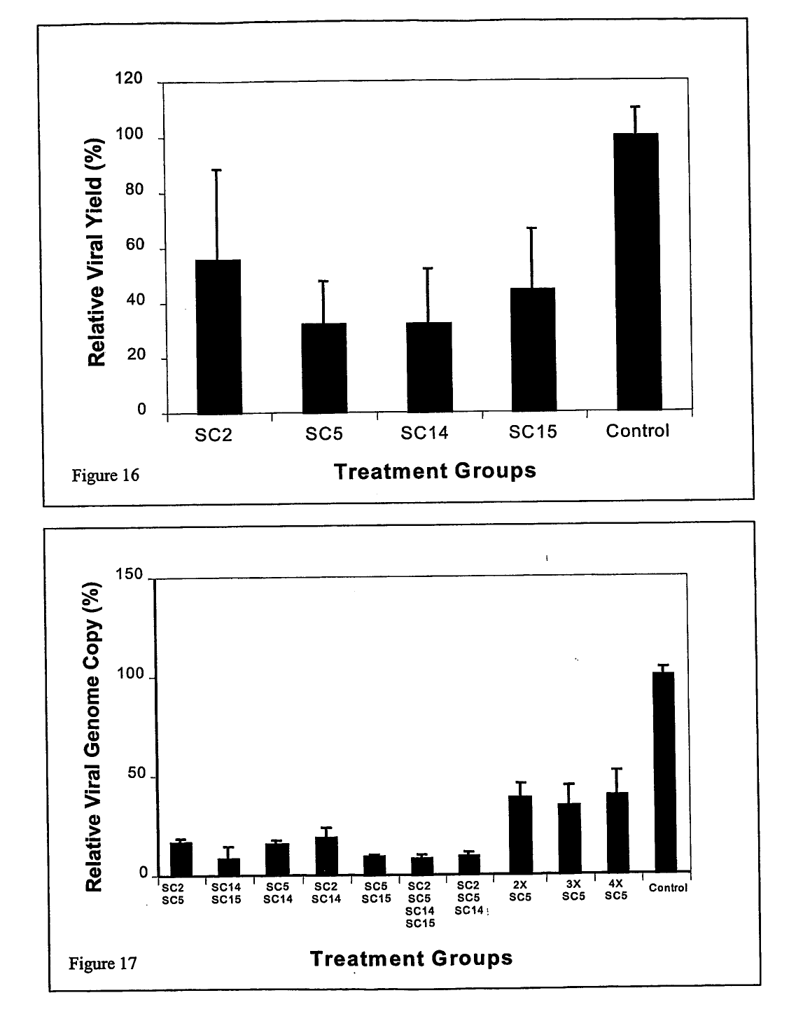

Twenty-four hours later, the cells and culture medium were collected for measurement of the cytoplasmic viral genome copy by Q-RT-PCR and viral titers by TCID₅₀. The relative viral genome copy numbers (FIG. 15) indicated that only one siRNA duplex, SC15, were able to achieve significant reduction. On the other hand, SC5 and SC14 were able to result in remarkable decreases of the viral titers (FIG. 16). Apparently, the siRNA-mediated therapeutic effects on the cells already infected by SARS-CoV were much weaker than the prophylactic effects.

These data imply that the weak effect of siRNA as a therapeutic agent might be due to the task of degrading preexisting viral genome RNA and sg mRNA, as well as the potential barrier to siRNA transfection caused by the viral infection. To improve the therapeutic effect of siRNA duplexes targeting SARS-CoV, the logical approaches are either to increase the dosage of the siRNA for transfection or to combine multiple siRNA duplexes targeting multiple regions of the viral genomic RNA. In the following experiments, the effort to enhance the therapeutic effect, and also the prophylactic effect, of the siRNA targeting SARS-CoV largely focused on those two approaches.

Combinational Effect With Multiple siRNA Duplexes

Although there have been many reports on siRNA-mediated antiviral activities by targeting single gene or single sequence region, limited evidence has been shown of the use of a combination of multiple siRNAs targeting various genes or regions. In an attempt to enhance the anti-SARS-CoV therapeutic effects, a strategy of combining multiple siRNA duplexes targeting different viral genes was evaluated. The siRNA combinations were chosen among the four selected active siRNA duplexes. The same dosage was used for the transfection regardless the number and composition of the siRNA species.

To test the hypothesis that combination of multiple active siRNA duplexes will improve the therapeutic effects, seven combinations of the active siRNAs at the same dosage were transfected into the cells already infected by the SARS-CoV. Thirty-six hours post transfection, the samples were collected for the measurement of viral genome copy number and viral titers. All combinations demonstrated much improved potency of the therapeutic effect: inhibition of viral replication for up to 80%, measured by viral genome copy number (FIG. 17). However, increasing 2 to 4 fold dosages of SC5, did not improve the inhibitory effect on SARS-CoV.

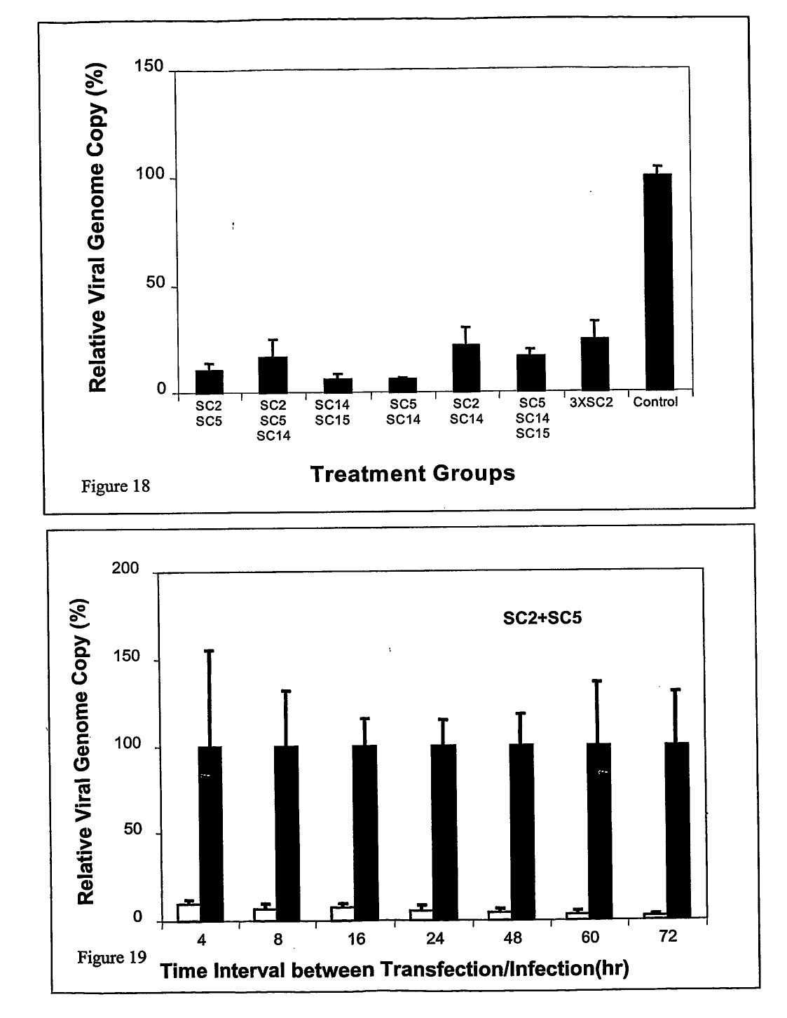

These data clearly demonstrated that combination of multiple siRNA duplexes targeting various regions of viral genomic RNA significantly enhanced the anti-SARS therapeutic effect, whereas increasing the dosage of the active siRNA duplexes might not have any significant impact. In addition, this result also suggested that SARS-CoV infection does not affect siRNA transfection and function. During the prophylactic effect study, transfection of various combinations of active siRNA duplexes was followed by SARS-CoV infection as described in the methods. Twenty-four hours later, the cells and culture medium were collected for Q-RT-PCT and TCID₅₀. Comparing the control samples, a significant reduction of the viral genome copy numbers was observed (FIG. 18) when cells were pretreated with various combinations of the active siRNA duplexes. Interestingly, increasing the dosage of SC2 (3×SC3) used in the study did not achieve significant improvement of the prophylactic effect. The prolonged prophylactic effect was also observed with combined siRNA duplexes, SC2 and SC5, for up to 72 hours (FIG. 19).

The siRNA-mediated inhibition of SARS-CoV replication observed in this study is likely due to capability of siRNA in disruption of the viral genomic RNA, in inactivation of the viral replication machinery and in reduction of the infectious virulence. Although the position effect of the siRNA within an open reading frame has been widely recognized [13, 16], the position effects of siRNA on the viral genome RNA has not been well appreciated. The study results indicated that the siRNA-mediated anti-SARS-CoV activity in non-human primate cell culture is both genome location-dependent and gene sequence-dependent. For example, the three most potent siRNA duplexes targeted the middle regions of the viral genome, and the SC2 and SC5 siRNAs targeted the first 50-200 nt of the open reading frames. The Spike specific siRNA, SC2, reduced both viral titer and viral genome copy number despite the biological role of Spike proteins is largely in viral infection.

It appears that reduction of viral genome copy number was the major effect of SC2 siRNA, instead of knockdown of Spike protein expression. This conclusion was also supported by the fact that among 32 siRNA duplexes targeting both the right hand ⅓ region of viral genome RNA and the various sg mRNAs, only SC2 presented the significant inhibitory effect. Apparently, the most siRNA duplexes targeting sg mRNAs played a little role in the viral replication inhibition. Therefore, the genomic RNA disruption may be the major function of these anti-SARS-CoV siRNAs,

Other Considerations

I. Key Proteins Required for Replications and Infections:

Since little is known about the new SARS coronavirus gene functions and genomic components at present time, genome structure information from a previously defined virus, Dengue fever virus (DEN), was used to identify open-reading frames for key proteins of the newly identified coronavirus genome sequences.

The DEN virus was chosen because DEN virus is similar to coronavirus in that they both are positive single-strand RNA virus, and it has been reported that DEN virus replication was inhibited by siRNA targeting of the prM gene of DEN virus. Based on the previously known information about the genome structure of DEN virus and published SARS coronavirus genome sequences, three putative open reading frames of key proteins were identified as targets for siRNA-mediated knockdown: nsp1, a processing enzyme for protein maturation; nsp9, an RNA dependent RNA polymerase and important for RNA genome replication and for production of sub-genomic mRNAs; and S protein (spike), a surface glycoprotein for receptor binding, cell fusion, induction of neutralizing antibody and cellular immunity.

II. Design of siRNA Duplexes

Template viral genome sequences: SARS-CUHM-WI (AY278554, GI:30023518) was used for selection of the specific siRNA duplexes targeting to the corresponding genes (open reading frames). The targeted genes are listed as following:

Targeted Genes:

nsp1: Coding for proteinase,

nsp9: Coding for RNA-dependent RNA Polymerase (RdRp), the sequence of SARS-CUHM-WI and SARS-Tor2 are identical.

S: Coding for spike protein that binds to cell receptor, induces fusion, and induces neutralizing Ab and T-cell immunity. There are 3 bp non-homologous to SARS-To2, which were avoided when designing siRNA duplexes.

Two siRNA duplexes were designated for each targeted genes based on the Tuschl's guidelines. The sequences and locations of these siRNA oligos are listed in the tables below. FIG. 2 also shows the map of the SARS coronavirus genome structure with the positions of the targeted sequences.

Table, Sequences of siRNA Targeting Coronavirus

All target sequences underwent a BLAST search for potential cross-talk to non-related sequences. The sequences shown below are all unique sequences that are homologous only to the published SARS coronavirus sequences including strains of SARS-Urbani and SARS-Tor2.

| | | |
|---|---|---|
| Genes | Targeted sequences (5′-3′) | Locations |

| | | | |
|---|---|---|---|
| nsp1 | 1 | AACCTTTGGAGAAGATACTGT | 2711-2731 nt |
| | 2 | AATCACATTTGAGCTTGATGA | 2762-2782 nt |
| nsp9 | 1 | AAGTTGCTGGTTTTGCAAAGT | 13467-13487 nt |
| | 2 | AAGGATGAGGAAGGCAATTTA | 13520-13540 nt |
| S | 1 | AAGCTCCTAATTACACTCAAC | 21543-21563 nt |
| (spike) | 2 | AATGTTACAGGGTTTCATACT | 21659-21679 nt |

Locations of Selected Targets in Virus Genomes

two siRNA duplexes were selected to target each of the putative open reading frames.

| SARS-CUHK | | Position on SARS-Urbani | Gene of SARS-Urbani |
|---|---|---|---|
| nsp1 | 1 | 2736-2756 nt | nsp-popyprotein |
| | 2 | 2787-2807 nt | pp1a/pp1ab |
| nsp9 | 1 | 13492-13512 nt | nsp-popyprotein |
| | 2 | 13545-13575 nt | pp1a/pp1ab |
| S(spike) | 1 | 21568-21588 nt | Spike Protein |
| | 2 | 21684-21704 nt | |

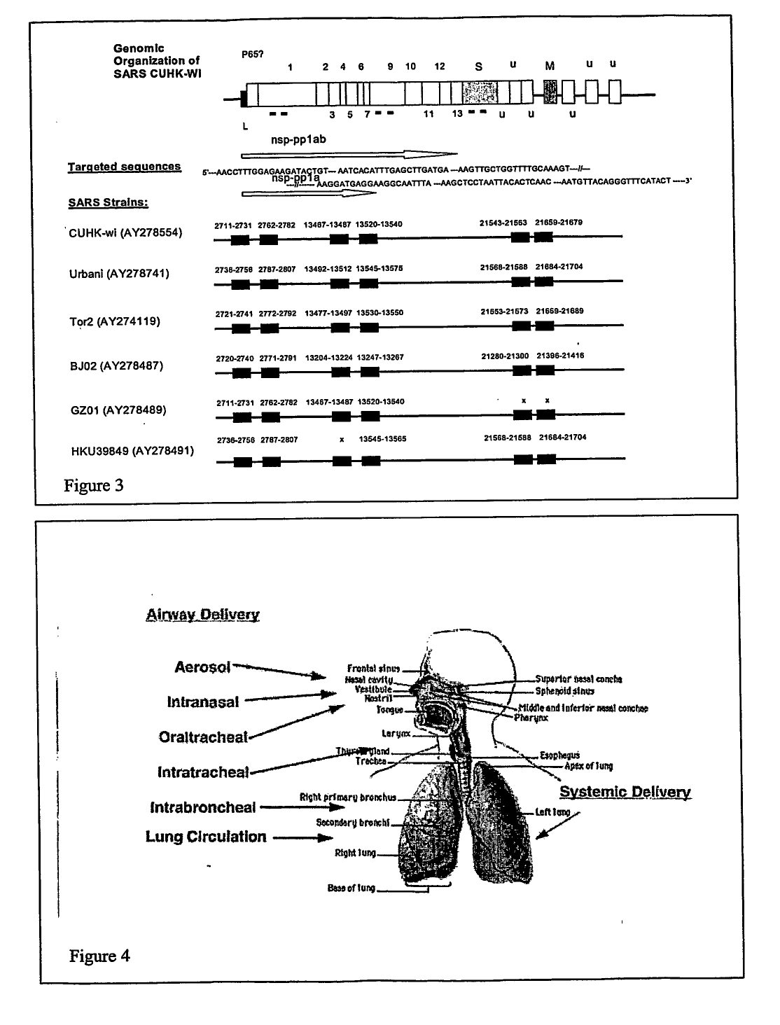

Additional SARS coronavirus sequences keep appearing in the public domains. The targeted sequences selected here have 100% homology to the most of those strains in the corresponding regions, except HKU39849 (FIG. 3).

Besides the above examples, other open reading frames and non-coding regions in the SARS coronavirus can also be targeted by specific RNAi agents for effective eradication of the coronavirus infection and replication.

III. RS-PCR for Detection of SARS Coronavirus

A unique RT-PCR assay called RNA template specific PCR(RS-PCR) has been designed for detection of SARS coronavirus RNA.

a. An RS-PCR based SARS diagnosis assay uses primers for detecting the SARS coronavirus sequences. Briefly, the assay uses a SARS coronavirus gene specific primer (SRT primer) which contains a 17 nt sequence complementary to the SARS coronavirus sequence and a special sequence of 30 nt attached to its 5′ for the reverse transcriptase (RT) synthesis of the first strand of cDNA from RNA of the SARS coronavirus genome. A pair of primers was then used for PCR amplification. The forward primer (Forw-primer) recognizes a sequence in the SARS coronavirus genome upstream of the 17 nt region recognize by the SRT primer. The reverse primer (Rev-primer) recognizes the special sequence attached to the SRT primer. The PCR amplification was performed at high annealing temperature (72° C.) at which only the cDNA from RT can be amplified but not any potential DNA contamination. The RS-PCR assay can be easily scaled up for large-scale application on diagnosis and prognosis.

b. RS-PCR Primers Design:

Primer 1: Forward-nsp1Up (30-mer, 41-70 nt of the putative nsp1 gene coding sequence, or 2734-2763 nt of coronavirus sequence, AY278554,)

5′---GGG AAG TTC AAG GTT ACA AGA ATG TGA GAA---3′

Primer 2: SRT-nsp1Dn (47-mer, the 17-mer at 3′ is complementary to 1041-1025 nt of the putative nsp1 gene coding sequence, or 3734-3718 nt of coronavirus sequence, AY278554). 5′ - - - GAA CAT CGA TGA CAA GCT TAG GTA TCG ATA gac aac ctg ctc ata aa - - - 3′

Primer3: Forward-nsp9Up (30-mer, 35-64 nt of the putative nsp9 gene coding sequence, or 13381-13410 nt of coronavirus sequence, AY278554). 5′ - - - CGG TGT AAG TGC AGC CCG TCT TAC ACC GTG - - - 3′

Primer4: SRT-nsp9Dn (47-mer, the 17-mer at 3′ is complementary to 734-718 nt of the putative nsp9 gene coding sequence, or 14080-14064 nt of coronavirus sequence, AY278554). 5′ - - - GAA CAT CGA TGA CAA GCT TAG GTA TCG ATA gag gat ggg cat cag ca - - - 3′

Primer5: Forward-SpikeUp (30-mer, 45-74 nt of coding sequence of the putative Spike gene coding sequence, or 21511-21540 nt of coronavirus sequence, AY278554). 5′ - - - CCT TGA CCG GTG CAC CAC TTT TGA TGA TGT - - - 3′

Primer6: SRT-SpikeDn (47-mer, the 17-mer at 3′ is complementary to 644-628 nt of the putative Spike gene coding sequence, or 22110-22094 nt of coronavirus sequence, AY278554). 5′ - - - GAA CAT CGA TGA CAA GCT TAG GTA TCG ATA gtg tta aaa cca gaa gg - - - 3′

Primer 7: (Rev-primer)

5′-AACATCGATGACAAGCTTAGGTATCGATA-3′

c. RS-PCR The following procedure is used for RS-PCR to detect SARS coronavirus in biological samples such as cell lysates, animal tissue and human patient tissue. Other tissues may also be used.

1). Total RNA was isolated from human sample using RNAwiz™ reagent (Ambion). MuLv Reverse Transcriptase and RNase inhibitor are available from Applied Biosystems and all other reagents used in the RS-PCR are available from PE Biosystems.

2). SRT reaction: 1 μg of total RNA sample was mixed with 2 μL of 10×PCRII buffer, 4 μl of 25 mM MgSO₄, 0.5 μl of 10 mM dNTPs, 1 μl RNase inhibitor (20 U/μl), 1 μl of 20 uM SRT primer, 1 μl of MuLv reverse transcriptase (50 U/μl), and RNase free water to a total volume of 20 μl. The sample was incubated at 37° C. for 30 minutes followed by at 42° C. for 15 minutes, then heated at 94° C. for 5 minutes.

3). PCR: 10 μl of SRT product was mixed with 4 μl of 10× PCRII buffer, 3 μl of 25 mM MgSO₄, 1 μl of 10 mM dNTPs, 1 μl of 20 uM Forw-primer, 1 μl of 20 uM Rev-primer, 0.5 μl of Taq DNA polymerase (5 U/μl), and distilled water to a total volume of 50 μl. The sample was heated at 94° C. for 2 minutes, and then subjected to 35 cycles of 2-step PCR: 94° C. for 1 minutes, annealing and extension at 72° C. for 2 minutes. An extra 10 minutes incubation at 72° C. was allowed at the end of PCR followed by incubation at 4° C., before the PCR products were analyzed by running 10 μl RS-PCR product in a 0.8% agarose gel.

TABLE
Lengths of RS-PCR Products

| | | Primer | Primers | Size of RS- |
|---|---|---|---|---|
| | Gene | For SRT | For PCR | PCR products |
| 1 | nsp1 | 2 | 1 + 7 | 1031 bp |
| 2 | nsp9 | 4 | 3 + 7 | 730 bp |
| 3 | S protein | 6 | 5 + 7 | 630 bp |

IV. Pulmonary siRNA Delivery

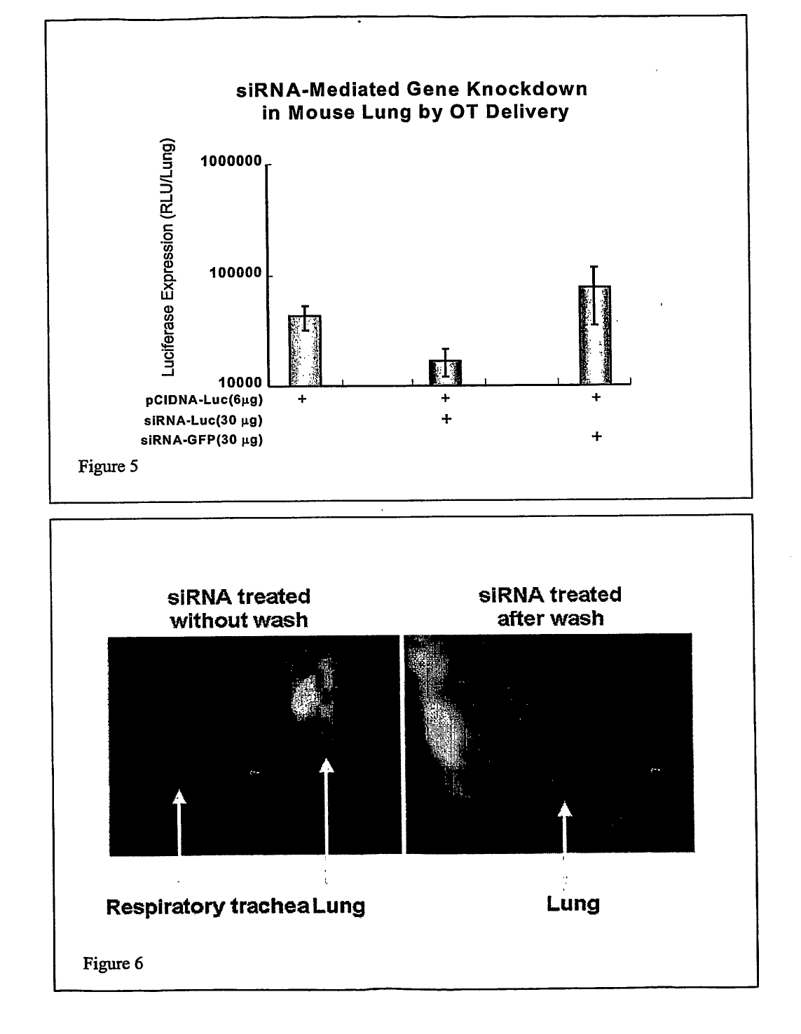

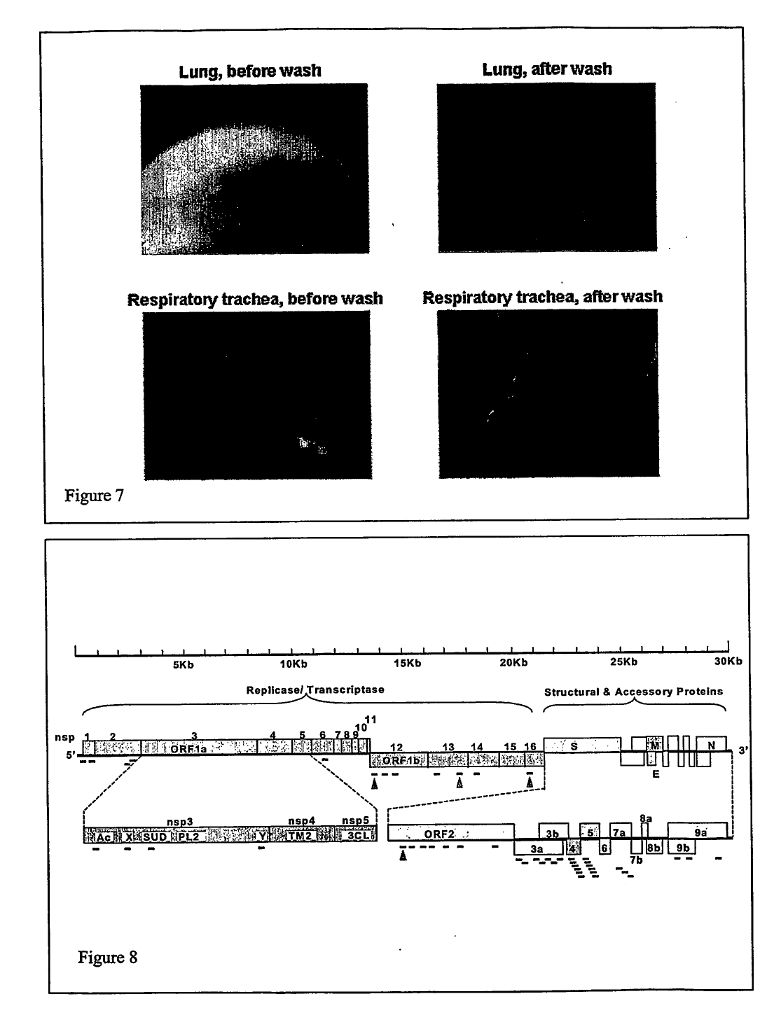

There are multiple routes for effective nucleic acid delivery into the mammalian airways (FIG. 4). We have developed an oral-tracheal delivery for siRNA duplex and other nucleic acid for effective gene expression manipulations (FIG. 5). The unique formulations related to this type of delivery include surfactant, liposome and peptide polymers. The nasal delivery and other types of airway delivery methods are also applied for achieving the most effective nucleic acid delivery. When fluorescence-labeled siRNA duplexes were administrated into the upper airway through nasal delivery and lower airway through oral-tracheal delivery, both trachea and lung were lighten up, even after the intensive wash (FIGS. 6 and 7).

Validation of the Active siRNA Molecules In Vivo

From the in vitro study of the 48 of SARS-specific siRNAs performed in 2003 [32], the following results were obtained: 1) Several effective siRNAs were selected that inhibited the SARS-CoV replication in the infected cells for up to 90%. 2) These effective siRNAs, when transfected into cells at 48-72 hours before viral infection, inhibited SARS-CoV viral replication for up to 90%, suggest a prophylactic effect of siRNA. 3) Simultaneous delivery of several siRNAs targeting different locations of the viral genome exhibited synergetic inhibitory effect. The pivotal step to demonstrate the safety of the selected siRNAs modality and their efficacy against SARS-CoV is to carry out an in vivo experiment in an established SARS animal model.

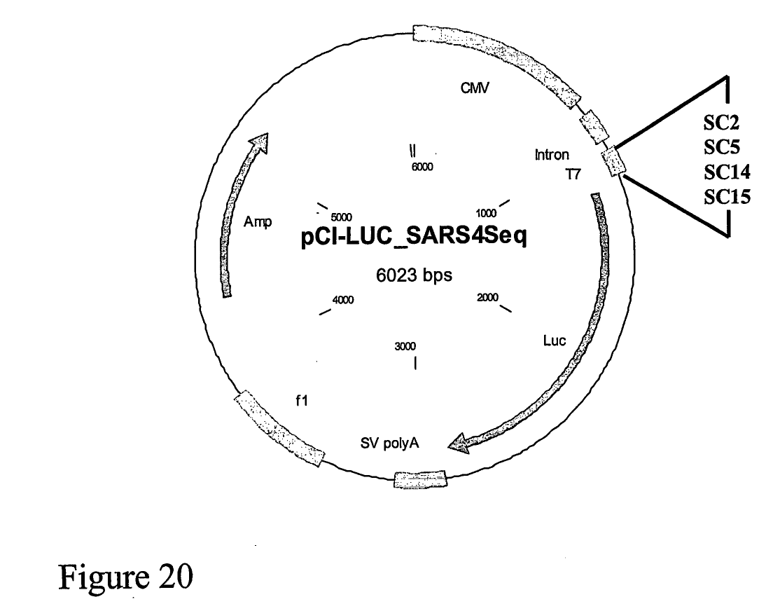

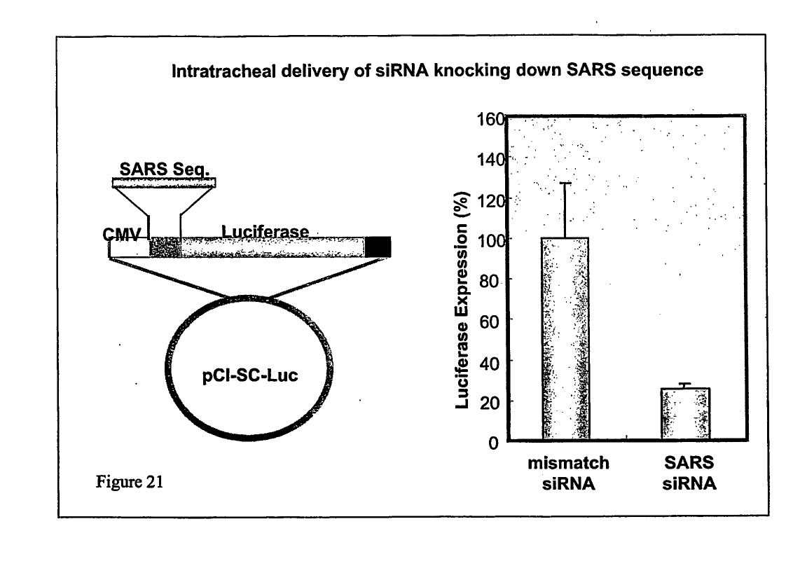

Before directly move the study of siRNA-mediated SARS-CoV inhibition in the non-human primate animal, we first constructed a surrogate plasmid by fusing a fragment of SARS-CoV sequence containing SC2 and SC5 targeted sequences with luciferase cDNA, pCI-Luc-SC (FIG. 20). When we co-transfected pCI-Luc-SC with SC2 and SC5 siRNA into the 293 cells, the Luciferase expression of the pCI-Luc-SC was significantly knocked down compared to the control siRNA duplexes (data not shown). After we confirmed that this surrogate approach worked well in vitro, we then co-delivered the plasmid and SC2-SC5 siRNA duplexes into the mouse lung through an intratracheal administration. Luciferase expression was significantly knocked down when the lung tissues were collected and the Luciferase activities were measured compared to the control siRNA (FIG. 21). This result provides strong support for the fact that siRNA is active in the animal airway and is able to knockdown the target gene expression.

Although some alternative animal models are being explored the non-human primate model remains the well-accepted standard simply because of its genetic and physiological similarities to human. The disease process of SARS consists of three phases: viral replication, immune hyperactivity, and pulmonary destruction; and the best period for siRNA modality to control the development of SARS disease is the first phase. Therefore, in the proposed in vivo experiment, we tested the efficacy of the siRNA modality at the early stage of the experimental SARS disease. To avoid the possibly intolerable toxicity that might be caused by high exposure to siRNA, we applied siRNA within 5 days post infection (p. i.) when multiple dosages were used. The main goal of this study was to test the efficacy of siRNAs against SARS, and to investigate the toxicity profile of the siRNA reagent at tested dosage in monkey model. The animal experiment and consequent assays were performed at the facility of the Institute of Laboratory Animal Science, CAMS (ILAS). All the experimental protocols will satisfy the relevant regulatory rules set up by the Ministry of Health of China.

Test System:

Animal, Virus Strain, and Animal Grouping:

A Rhesus monkey SARS model system was established by ILAS. This model showed infection of monkeys by SARS-CoV strain isolated from SARS patients in China. The infected monkeys developed SARS-like symptoms, pathology, and hematological profile. We will use the same SARS-CoV strain employed by the ILAS to challenge the monkeys, and delivery siRNAs into the respiratory tract. In the first experiment, 5 groups of animal (a total of 20 monkeys) are used, (Table 1). The principle of the grouping is: Group 1 (G1) is set for observation of prophylactic effect, G2 and G3 for therapeutic effect, with a difference in whether the first dosage of siRNA is applied at the same time of viral infection of not. G4 serves as a therapeutic siRNA control using unrelated siRNA, and G5 is the untreated group, the healthy animals being challenged with virus. Table 2 summarizes the total amount of siRNA used.

Virus Challenging:

SARS CoV is administrated via nasal inhalation and spray, as selected by the ILAS through comparison studies of different delivery routes.

Delivery of siRNAs:

siRNAs are mixed with an appropriate volume of dissolving solution (5% glucose in RNase-free water). Although there is no reference available for the effective delivery of siRNA into monkey lungs, a recent mouse study indicated that lung-specific siRNA delivery could be achieved by intranasal administration without the need for viral vectors or transfection agents. We deliver siRNA solution through nasal inhalation and spray, the same as that used for viral challenging.

TABLE 1
Treatment Groups

| | | No. | | Amount | Appli- |
|---|---|---|---|---|---|
| | | Ani- | | per | cation |
| Groups | Description | mal | Payloads | dosage | time |
| G1 | High dose/ | 4 | siRNA.SARS.Mix | 30 mg/ | 4 hrs |
| | Prophylactic | | | animal | before |
| | | | | | infection |
| G2 | High dose/ | 4 | siRNA.SARS.Mix | 30 mg/ | 0, 24, |
| | Therapeutic. | | | animal | 72, 120 |
| | | | | | hrs p.i. |
| G3 | High dose/ | 4 | siRNA.SARS.Mix | 30 mg/ | 4, 24, 72, |
| | Therapeutic | | | animal | hrs p.i. |
| G4 | High dose/ | 4 | siRNA.Luc | 30 mg/ | 0, 24, 72, |
| | Therapeutic, | | | animal | 120 hrs |
| | siRNA | | | | p.i. |
| | Control | | | | |
| G5 | Untreated | 4 | No siRNA | | |

TABLE 2
Reagents used

| | | | | | | | | | | Subtotal |
|---|---|---|---|---|---|---|---|---|---|---|
| siRNA | G1 | | G2 | | G3 | | G4 | | G5 | Amount |
| siSC2 | 60 | mg | 240 | mg | 180 | mg | | | | 480 mg |
| siSC5 | 60 | mg | 240 | mg | 180 | mg | | | | 480 mg |
| siLuc | | | | | | | 480 | mg | | 480 mg |
| Subtotal | 120 | | 480 | | 360 | | 480 | | 0 | 1440 mg |

Evaluation of the Efficacy and Toxicity of siRNA

The efficacy of siRNA against SARS is reflected by the inhibitory effect of siRNA on SARS-CoV virus replication, symptom, pathology and physiological index. The toxicity of siRNA mostly is shown by clinical signs and/or pathology, basically reflected by the tolerability of animals to the applied siRNA dosage.

Replication of SARS-CoV virus:

On day 0 (right before challenge), 4, and 7 p.i., monkeys are anaesthetized and nasal/pharyngeal swabs and blood samples are taken. Swabs are used for detection of viral genomic mRNA copy number (by real time quantitative RT-PCR, Q-RT-PCR) and isolation of SARS-CoV (through infection of permissive cells); blood sample for Q-RT-PCR. When monkeys are sacrificed (two on day 7, and two on day 11, p.i.), lungs and blood samples are collected for viral isolation. The primers used for Q-RT-PCR were previously described. Viral isolation is performed by infecting permissive cells (e.g., Vero cells) with the swab or blood samples as specified above. CPE may appear after 1 to 3 blind passages on tissue culture. CPE will be recorded, and supernatant of tissue culture of each passage will be tested by Q-RT-PCR. Based upon the CPE appearance, some tissue culture supernatant samples of same passage will be compared for the viral titer indicated as TCID50. This hopefully will show some dynamic difference between tested and control groups. As a reference, the Q-RT-PCR assay could detect 89% of the 89% SARS patients, and viral isolation may take more than one run of passage in tissue culture.

Clinical sign and function of lung: are recorded daily, including respiratory symptoms, body temperature, size of tracheobronchial lymph nodes. Additionally, the analysis of arterial blood, and pulse oximetry are also measured.

Histological tests: On day 7 and day 11, p.i., two monkeys of each group, are sacrificed, respectively. Lung tissue sections are subjected to traditional histological and immunohistological tests (including in situ hybridization, FISH).

Routine blood tests: Blood samples are taken at the same time the swabs are taken. The major items in routine blood test are to be measured, e.g., WBC, DC, RBC, GB, HCT, MCV, MCH, and RDW. Liver enzymeactivity tests: Routine liver activity tests are to be performed, e.g., serum ALT, serum bilirubin, prothrumbin, albumin, LDH, etc.

Preliminary Results:

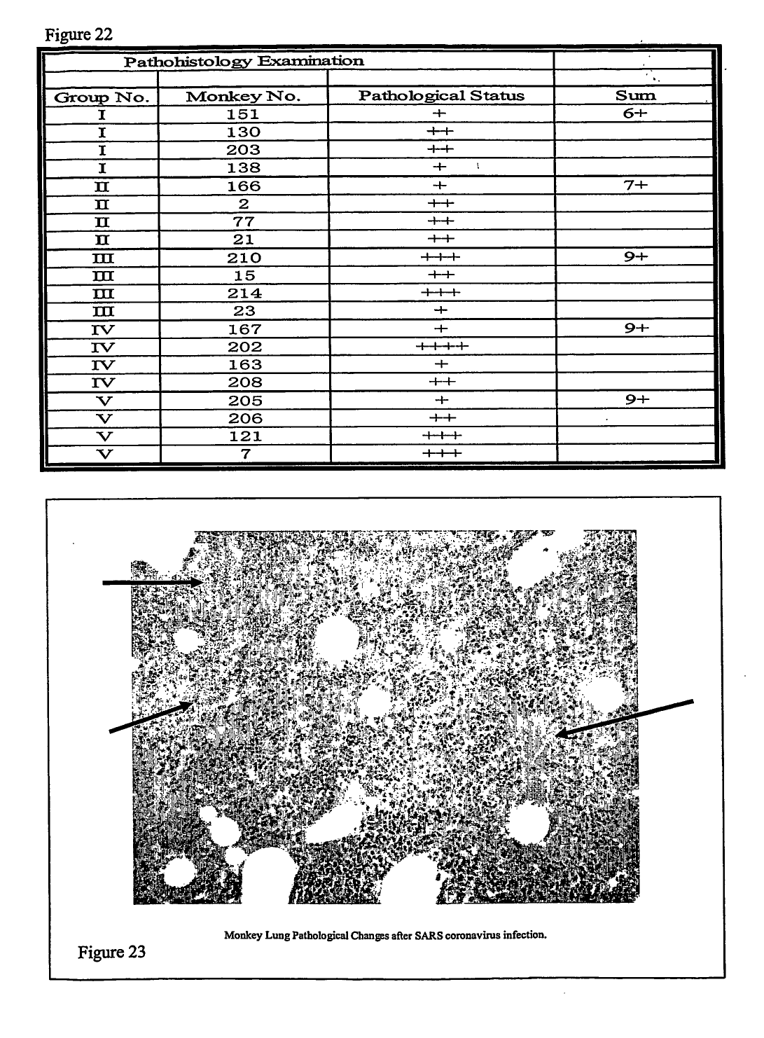

Delivery of siRNA duplex with the dosage of 30 mg per dose and 4 repeated dosing is safe. None of the treated monkey developed visible symptoms after the dosing, and no damage of the treated monkey lungs caused by siRNA delivery rather than SARS-CoV infection. Repeated intranasal delivery of 0.5 ml solution containing the siRNA drug into monkey lung was very effective (FIG. 22). The prophylactic effect of the siRNA duplexes in the monkey lungs were observed according to the comparison of animal lung pathological status (FIG. 23) between Group I and Group IV or V.

Co-administration of the siRNA duplexes and SARS-CoV into the monkey lungs resulted in anti-SARS-CoV activity according to the comparison of animal lung pathological status between Group I and Group IV or V. Clearly, the anti-SARS-CoV activities we observed in the cell culture study were further confirmed in this monkey study. These results demonstrate that siRNA-based anti-SARS-CoV therapeutics are effective for SARS treatment with high specificity and safety.

### REFERENCES

1) Peiris J. S, et al., 2003. Lancet 361, 1319-1325.
2) Ksiazed, T. G., et al., 2003. New Eng. J. Med. 348, 1953-1966.
3) Drosten, C., et al., 2003. New Eng. J. Med. 348, 1-10.
4) Fouchier R. A. M., et al., 2003. Nature. 423, 240.
5) Zhong, N. S., et al., 2004. Lancet (in press).
6) Fire A, et al., 1998. Nature 391: 806-811.
7) Bernstein, E., et al., Nature 409, 363-366.
8) Hammond, S. M., et al., 2000. Nature 404, 293-296.
9) McManus, M. T. and Sharp, P. A., 2002. Nature Reviews, Genetics. 3, 737.
10) Tuschl, T., 2002. Nature Biotechnology, 20, 446-448.
11) Bushman, F., 2003. Molecular Therapy 7, 9-10.
12) Lu, Y. P., et al., 2003. Current Opinion in Molecular Therapeutics, 5, 225-234.
13) Jacque, J. M., et al., Nature 418, 435-438.
14) Lee, N. S., et al., 2002. Nat. Biotechnol. 20, 500-5.
15) Novina, C. D., et al., 2002. Nat. Med. 8, 681-6.
16) Ge, Q., et al., 2003. Proc Natl Acad Sci USA. 100, 2718-23.
17) Hu, W., et al., 2002. Current Biol. 12, 1301-11.
18) Gitlin, L., et al., 2002. Nature 418, 430-434.
19) Shlomai, A., and Shaul, Y., 2003. Hepatology 37, 764-770.
20) Jiang, M., and Milner, J., 2002. Oncogene 21, 6041-6048.
21) Coburn, G. A., and Cullen, B. R., 2002. J. Virol. 76, 9225-9231.
22) McCaffrey, A. P., et al., 2003. Nature Biotechnology, 21, 639-644.
23) Klein, C., et al., 2003. Gastroenterology 125, 9-18.
24) Peiris J S, et al., 2003, Lancet 361(9371): 1767-72
25) Elbashir, S. M., et al., 2001. EMBO J. 20, 6877-6888.
26) Stevenson M. 2003, Nature Reviews, Immunology, 3:851-858.
27) Rota, P. A. et al., 2003. Science 300, 1394-1399.
28) Marra, M. A. et al., 2003. Science 300, 1399-1404.
29) Snijder E J et al, 2003, J. Mol. Biol. 331:991-1004.
30) Bitio V, and S. Barik, 2001, BMC Microbiol 1(1):34
31) Dykxhoorn D M, 2003, Nature Review, Molecular Cell Biology, 4:457-467
32) Bo-jian Zheng, et al 2004. Antiviral Therapy, (Accepted for June 2004 publication).

1. An isolated double stranded RNA molecule comprising a first strand comprising a ribonucleotide sequence which corresponds to a nucleotide sequence of a SARS virus and a second strand comprising a ribonucleotide sequence which is complementary to said nucleotide sequence of said SARS virus, wherein said double-stranded molecule inhibits expression of said nucleotide sequence of said SARS virus.

2. The RNA molecule according to claim 1 wherein said first and second strands are separate complementary strands.

3. The RNA molecule according to claim 1 wherein said first and second strands are contained in a single molecule, wherein said single molecule comprises a loop structure.

4. The RNA molecule according to any preceding claim wherein said nucleotide sequence of a SARS virus is selected from the group consisting of an nsp1 sequence, an nsp9 sequence and a spike sequence.

5. The RNA molecule according to claim 4, wherein said first strand comprises a sequence selected from the group consisting of

| | |
|---|---|
| AACCTTTGGAGAAGATACTGT, | (SEQ ID NO: 1) |
| AATCACATTTGAGCTTGATGA, | (SEQ ID NO: 2) |
| AAGTTGCTGGTTTTGCAAAGT, | (SEQ ID NO: 3) |
| AAGGATGAGGAAGGCAATTTA, | (SEQ ID NO: 4) |
| AAGCTCCTAATTACACTCAAC, | (SEQ ID NO: 5) |
| and | |
| AATGTTACAGGGTTTCATACT. | (SEQ ID NO: 6) |

6. A method of detecting a SARS virus in a sample, comprising (a) contacting RNA obtained from said sample with a gene specific primer comprising a 3′ region that is complementary to a SARS sequence and a 5′ sequence that is not complementary to a SARS sequence and synthesizing a first strand cDNA molecule by reverse transcription followed by (b) amplifying said first strand cDNA in a PCR using a pair of primers, wherein the first primer is complementary to said 5′ region of said gene specific primer and wherein the second primer comprises a sequence in the SARS genome that is upstream of the region recognized by said 3′ region of said gene specific primer, and (c) detecting the product of said PCR.

7. The method of claim 6 wherein said gene specific primer is complementary to a SARS nps1, nps9 or spike sequence.

8. The method of claim 7, wherein said gene specific primer comprises a sequence is selected from the group consisting of

| | |
|---|---|
| GAA CAT CGA TGA CAA GCT TAG GTA TCG | (SEQ ID NO: 7) |
| ATA gac aac ctg ctc ata aa, | |
| GAA CAT CGA TGA CAA GCT TAG GTA TCG | (SEQ ID NO: 8) |
| ATA gag gat ggg cat cag ca, | |
| and | |
| CAA CAT CGA TGA CAA GCT TAG GTA TCG | (SEQ ID NO: 9) |
| ATA gtg tta aaa cca gaa gg. | |

9. The method of claim 8, wherein said first primer comprises the sequence

| | |
|---|---|
| GAACATCGATGACAAGCTTAGGTATCGATA | (SEQ ID NO: 10) |

10. The method of claim 6 wherein said second primer comprises a sequence selected from the group consisting of

| | |
|---|---|
| GGG AAG TTC AAG GTT ACA AGA ATG TGA | (SEQ ID NO: 11) |
| GAA, | |
| CGG TGT AAG TGC AGC CCG TCT TAC ACC | (SEQ ID NO: 12) |
| GTG, | |
| and | |
| CCT TGA CCG GTG CAC CAC TTT TGA TGA | (SEQ ID NO: 13) |
| TGT. | |

11. A method of treating or preventing a coronavirus infection in a subject, comprising administering to said subject and effective amount of a composition comprising an isolated double stranded RNA molecule, wherein said RNA molecule comprises a first strand comprising a ribonucleotide sequence which corresponds to a nucleotide sequence of a coronavirus and a second strand comprising a ribonucleotide sequence which is complementary to said nucleotide sequence of said coronavirus, wherein said double-stranded molecule inhibits expression of said nucleotide sequence of said coronavirus.

12. The method according to claim 11, wherein said coronavirus is a SARS virus.

13. The method according to claim 12, wherein said first and second strands are separate complementary strands.

14. The method according to claim 12, wherein said first and second strands are contained in a single molecule, wherein said single molecule comprises a loop structure.

15. The method according to claim 12 wherein said nucleotide sequence of a SARS virus is selected from the group consisting of an nsp1 sequence, an nsp9 sequence and a spike sequence.

16. The method according to claim 15, wherein said first strand comprises a sequence selected from the group consisting of

| | |
|---|---|
| AACCTTTGGAGAAGATACTGT, | (SEQ ID NO: 1) |
| AATCACATTTGAGCTTGATGA, | (SEQ ID NO: 2) |
| AAGTTGCTGGTTTTGCAAAGT, | (SEQ ID NO: 3) |
| AAGGATGAGGAAGGCAATTTA, | (SEQ ID NO: 4) |
| AAGCTCCTAATTACACTCAAC, | (SEQ ID NO: 5) |
| and | |
| AATGTTACAGGGTTTCATACT. | (SEQ ID NO: 6) |

17. The method according to claim 15 wherein said double stranded RNA molecule comprises a sequence selected from the group consisting of SC2, SC5, SC14 and SC15.

18. The method according to claim 12, wherein said double stranded RNA molecule is delivered into the airway of said subject.

19. The method according to claim 18, wherein said delivery into said airway is achieved by intranasal delivery or by delivery into the trachea.

20. The method according to claim 12, wherein said composition comprises said double stranded RNA molecule in a carrier comprising an aqueous glucose solution free of RNAse.

21. The method according to claim 20 wherein said glucose solution comprises about 5% glucose.

22. The method according to claim 12, wherein the dosage of said double stranded RNA molecule is 1-100 mg per kg of body weight of said subject.

23. The method according to claim 12, wherein said composition is delivered as an aqueous RNA-free solution, in an aerosol or in a powder.

24. A method of treating a respiratory disease in a subject, comprising administering to the airway of said subject a double stranded RNA molecule comprising a first strand comprising a ribonucleotide sequence which corresponds to a nucleotide sequence of a gene implicated in said disease and a second strand comprising a ribonucleotide sequence which is complementary to said nucleotide sequence of said nucleotide sequence of said gene, wherein said gene implicated in said disease exhibits undesirably high levels of gene expression in said disease, and wherein said double-stranded molecule inhibits expression of said nucleotide sequence of said gene implicated in said disease.

25. The method according to claim 24, wherein said gene implicated in said disease is a gene of a pathogenic organism.

26. The method according to claim 25, wherein said pathogenic organism is a bacterium, a virus or a fungus.

27. The method according to claim 24, wherein said disease is autoimmune inflammation or lung cancer.

28. The method according to claim 12, wherein at least two double stranded RNA molecules targeting at least two different nucleotide sequences of a SARS virus are used.

29. The method according to claim 28 wherein said two nucleotide sequences are selected from the group consisting of an nsp1 sequence, an nsp9 sequence and a spike sequence.
